(19) United States
(12) Patent Application Publication
(10) Pub. No.: US 2004/0105778 A1
(43) Pub. Date: Jun. 3, 2004

(54) Gamma irradiation of solid nanoparticulate active agents

(75) Inventors: Robert Lee, Boyertown, PA (US); Matthew Hilborn, Spring City, PA (US); Laura Kline, Harleysville, PA (US); Janine Keller, Collegeville, PA (US)

Correspondence Address: FOLEY AND LARDNER, SUITE 500, 3000 K STREET NW, WASHINGTON, DC, 20007, US

(73) Assignee: Elan Pharma International Limited

(21) Appl. No.: 10/654,600

(22) Filed: Sep. 4, 2003

Related U.S. Application Data
(60) Provisional application No. 60/415,749, filed on Oct. 4, 2002.

(51) Int. Cl. A61L 9/00; B01J 19/00; A62B 7/08; B01J 19/08; A61L 2/00

(52) U.S. Cl. 422/22; 422/1; 422/4; 422/28; 422/40; 422/121; 422/123; 422/186; 422/292; 422/300; 422/305; 422/306

(57) ABSTRACT

The present invention relates to methods for terminal sterilization of solid forms of nanoparticulate active agent compositions via gamma irradiation. The nanoparticulate active agent has an effective average particle size of less than about 2 microns, prior to incorporation into a solid form for sterilization. The resultant sterilized compositions exhibit excellent redispersibility, homogeneity, and uniformity. Also encompassed are compositions made via the described method and methods of treating animals and humans using such compositions.

### FIELD OF THE INVENTION

The present invention relates to methods for terminal sterilization of nanoparticulate active agent compositions via gamma irradiation. Specifically, it is related to methods of terminal sterilization of a solid comprising a nanoparticulate active agent. Preferably the active agent has an effective average particle size of less than about 2 microns. The resultant radiated nanoparticulate active agent compositions exhibit excellent particle redispersibility, homogeneity, and uniformity.

### BACKGROUND OF THE INVENTION

A. Background Regarding Gamma Irradiation

Irradiating a product with gamma radiation is one method of sterilizing a pharmaceutical product. Gamma irradiation is effective in destroying viruses and bacteria when given in high total doses. Unlike other sterilization methods, such as ethylene oxide sterilization, radiation sterilization has the advantages of high penetrating ability and instantaneous effects, without the need to control temperature, pressure, vacuum, or humidity.

U.S. Pat. No. 4,330,626 describes a process of preparing urease from jack beans. As part of the process, the beans are irradiated to reduce microbial contamination. The irradiation of the beans occurs prior to any size reduction of the seeds of the jack beans. This is done because there is loss of activity of the urease by irradiating the beans after particle size reduction.

U.S. Pat. No. 6,066,292 describes the sterilization of pharmaceuticals including a suspension by a technique other than gamma radiation. Gamma irradiation is generally discussed in the background of the invention.

U.S. Pat. No. 6,607,695 describes a method of sterilizing a chemical composition contained in a sealed container comprising exposing the container to gamma radiation. U.S. Pat. No. 6,596,230 relates to the treatment of biological fluids with sterilizing radiation, such as gamma radiation, to inactivate various pathogens, such as viruses, in a continuous flow arrangement while exhibiting radiation dose uniformity. Similarly, U.S. Pat. No. 6,346,216 relates to a method for sterilizing biological products to inactivate biological contaminants, such as viruses, bacteria, yeasts, molds, mycoplasmas, and parasites, comprising irradiating the product with gamma radiation at a low dose rate from about 0.1 kGy/hr to about 3.0 kGy/hr for a period of time sufficient to sterilize the product. U.S. Pat. No. 6,524,528 describes a method of sterilizing a tattooing solution, such as an india ink solution, through irradiation, such as gamma radiation.

B. Background Regarding Nanoparticulate Compositions

Nanoparticulate compositions, first described in U.S. Pat. No. 5,145,684 (“the '684 patent”), are particles consisting of a poorly soluble active agent having adsorbed onto the surface thereof a non-crosslinked surface stabilizer. The '684 patent also describes methods of making such nanoparticulate compositions. Nanoparticulate compositions are desirable because with a decrease in particle size, and a consequent increase in surface area, a composition is rapidly dissolved and absorbed following administration. The '684 patent does not teach or suggest sterilization of nanoparticulate compositions via gamma irradiation.

Methods of making nanoparticulate compositions are described, for example, in U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles.”

Nanoparticulate compositions are also described, for example, in U.S. Pat. No. 5,298,262 for “Use of Ionic Cloud Point Modifiers to Prevent Particle Aggregation During Sterilization;” U.S. Pat. No. 5,302,401 for “Method to Reduce Particle Size Growth During Lyophilization;” U.S. Pat. No. 5,318,767 for “X-Ray Contrast Compositions Useful in Medical Imaging;” U.S. Pat. No. 5,326,552 for “Novel Formulation For Nanoparticulate X-Ray Blood Pool Contrast Agents Using High Molecular Weight Non-ionic Surfactants;” U.S. Pat. No. 5,328,404 for “Method of X-Ray Imaging Using Iodinated Aromatic Propanedioates;” U.S. Pat. No. 5,336,507 for “Use of Charged Phospholipids to Reduce Nanoparticle Aggregation;” U.S. Pat. No. 5,340,564 for “Formulations Comprising Olin 10-G to Prevent Particle Aggregation and Increase Stability;” U.S. Pat. No. 5,346,702 for “Use of Non-Ionic Cloud Point Modifiers to Minimize Nanoparticulate Aggregation During Sterilization;” U.S. Pat. No. 5,349,957 for “Preparation and Magnetic Properties of Very Small Magnetic-Dextran Particles;” U.S. Pat. No. 5,352,459 for “Use of Purified Surface Modifiers to Prevent Particle Aggregation During Sterilization;” U.S. Pat. Nos. 5,399,363 and 5,494,683, both for “Surface Modified Anticancer Nanoparticles;” U.S. Pat. No. 5,401,492 for “Water Insoluble Non-Magnetic Manganese Particles as Magnetic Resonance Enhancement Agents;” U.S. Pat. No. 5,429,824 for “Use of Tyloxapol as a Nanoparticulate Stabilizer;” U.S. Pat. No. 5,447,710 for “Method for Making Nanoparticulate X-Ray Blood Pool Contrast Agents Using High Molecular Weight Non-ionic Surfactants;” U.S. Pat. No. 5,451,393 for “X-Ray Contrast Compositions Useful in Medical Imaging;” U.S. Pat. No. 5,466,440 for “Formulations of Oral Gastrointestinal Diagnostic X-Ray Contrast Agents in Combination with Pharmaceutically Acceptable Clays;” U.S. Pat. No. 5,470,583 for “Method of Preparing Nanoparticle Compositions Containing Charged Phospholipids to Reduce Aggregation;” U.S. Pat. No. 5,472,683 for “Nanoparticulate Diagnostic Mixed Carbamic Anhydrides as X-Ray Contrast Agents for Blood Pool and Lymphatic System Imaging;” U.S. Pat. No. 5,500,204 for “Nanoparticulate Diagnostic Dimers as X-Ray Contrast Agents for Blood Pool and Lymphatic System Imaging;” U.S. Pat. No. 5,518,738 for “Nanoparticulate NSAID Formulations;” U.S. Pat. No. 5,521,218 for “Nanoparticulate Iododipamide Derivatives for Use as X-Ray Contrast Agents;” U.S. Pat. No. 5,525,328 for “Nanoparticulate Diagnostic Diatrizoxy Ester X-Ray Contrast Agents for Blood Pool and Lymphatic System Imaging;” U.S. Pat. No. 5,543,133 for “Process of Preparing X-Ray Contrast Compositions Containing Nanoparticles;” U.S. Pat. No. 5,552,160 for “Surface Modified NSAID Nanoparticles;” U.S. Pat. No. 5,560,931 for “Formulations of Compounds as Nanoparticulate Dispersions in Digestible Oils or Fatty Acids;” U.S. Pat. No. 5,565,188 for “Polyalkylene Block Copolymers as Surface Modifiers for Nanoparticles;” U.S. Pat. No. 5,569,448 for “Sulfated Non-ionic Block Copolymer Surfactant as Stabilizer Coatings for Nanoparticle Compositions;” U.S. Pat. No. 5,571,536 for “Formulations of Compounds as Nanoparticulate Dispersions in Digestible Oils or Fatty Acids;” U.S. Pat. No. 5,573,749 for “Nanoparticulate Diagnostic Mixed Carboxylic Anydrides as X-Ray Contrast Agents for Blood Pool and Lymphatic System Imaging;” U.S. Pat. No. 5,573,750 for “Diagnostic Imaging X-Ray Contrast Agents;” U.S. Pat. No. 5,573,783 for “Redispersible Nanoparticulate Film Matrices With Protective Overcoats;” U.S. Pat. No. 5,580,579 for “Site-specific Adhesion Within the GI Tract Using Nanoparticles Stabilized by High Molecular Weight, Linear Poly(ethylene Oxide) Polymers;” U.S. Pat. No. 5,585,108 for “Formulations of Oral Gastrointestinal Therapeutic Agents in Combination with Pharmaceutically Acceptable Clays;” U.S. Pat. No. 5,587,143 for “Butylene Oxide-Ethylene Oxide Block Copolymers Surfactants as Stabilizer Coatings for Nanoparticulate Compositions;” U.S. Pat. No. 5,591,456 for “Milled Naproxen with Hydroxypropyl Cellulose as Dispersion Stabilizer;” U.S. Pat. No. 5,593,657 for “Novel Barium Salt Formulations Stabilized by Non-ionic and Anionic Stabilizers;” U.S. Pat. No. 5,622,938 for “Sugar Based Surfactant for Nanocrystals;” U.S. Pat. No. 5,628,981 for “Improved Formulations of Oral Gastrointestinal Diagnostic X-Ray Contrast Agents and Oral Gastrointestinal Therapeutic Agents;” U.S. Pat. No. 5,643,552 for “Nanoparticulate Diagnostic Mixed Carbonic Anhydrides as X-Ray Contrast Agents for Blood Pool and Lymphatic System Imaging;” U.S. Pat. No. 5,718,388 for “Continuous Method of Grinding Pharmaceutical Substances;” U.S. Pat. No. 5,718,919 for “Nanoparticles Containing the R(−)Enantiomer of Ibuprofen;” U.S. Pat. No. 5,747,001 for “Aerosols Containing Beclomethasone Nanoparticle Dispersions;” U.S. Pat. No. 5,834,025 for “Reduction of Intravenously Administered Nanoparticulate Formulation Induced Adverse Physiological Reactions;” U.S. Pat. No. 6,045,829 “Nanocrystalline Formulations of Human Immunodeficiency Virus (HIV) Protease Inhibitors Using Cellulosic Surface Stabilizers;” U.S. Pat. No. 6,068,858 for “Methods of Making Nanocrystalline Formulations of Human Immunodeficiency Virus (HIV) Protease Inhibitors Using Cellulosic Surface Stabilizers;” U.S. Pat. No. 6,153,225 for “Injectable Formulations of Nanoparticulate Naproxen;” U.S. Pat. No. 6,165,506 for “New Solid Dose Form of Nanoparticulate Naproxen;” U.S. Pat. No. 6,221,400 for “Methods of Treating Mammals Using Nanocrystalline Formulations of Human Immunodeficiency Virus (HIV) Protease Inhibitors;” U.S. Pat. No. 6,264,922 for “Nebulized Aerosols Containing Nanoparticle Dispersions;” U.S. Pat. No. 6,267,989 for “Methods for Preventing Crystal Growth and Particle Aggregation in Nanoparticle Compositions;” U.S. Pat. No. 6,270,806 for “Use of PEG-Derivatized Lipids as Surface Stabilizers for Nanoparticulate Compositions;” U.S. Pat. No. 6,316,029 for “Rapidly Disintegrating Solid Oral Dosage Form,” U.S. Pat. No. 6,375,986 for “Solid Dose Nanoparticulate Compositions Comprising a Synergistic Combination of a Polymeric Surface Stabilizer and Dioctyl Sodium Sulfosuccinate;” U.S. Pat. No. 6,428,814 for “Bioadhesive Nanoparticulate Compositions Having Cationic Surface Stabilizers;” U.S. Pat. No. 6,431,478 for “Small Scale Mill;” U.S. Pat. No. 6,432,381 for “Methods for Targeting Drug Delivery to the Upper and/or Lower Gastrointestinal Tract;” U.S. Pat. No. 6,582,285 for “Apparatus for Sanitary Wet Milling;” and U.S. Pat. No. 6,592,903 for “Nanoparticulate Dispersions Comprising a Synergistic Combination of a Polymeric Surface Stabilizer and Dioctyl Sodium Sulfosuccinate;” all of which are specifically incorporated by reference. In addition, U.S. patent application Ser. No. 20,020,012,675 A1, published on Jan. 31, 2002, for “Controlled Release Nanoparticulate Compositions,” describes nanoparticulate compositions, and is specifically incorporated by reference.

Amorphous small particle compositions are described, for example, in U.S. Pat. No. 4,783,484 for “Particulate Composition and Use Thereof as Antimicrobial Agent;” U.S. Pat. No. 4,826,689 for “Method for Making Uniformly Sized Particles from Water-Insoluble Organic Compounds;” U.S. Pat. No. 4,997,454 for “Method for Making Uniformly-Sized Particles From Insoluble Compounds;” U.S. Pat. No. 5,741,522 for “Ultrasmall, Non-aggregated Porous Particles of Uniform Size for Entrapping Gas Bubbles Within and Methods;” and U.S. Pat. No. 5,776,496, for “Ultrasmall Porous Particles for Enhancing Ultrasound Back Scatter.” None of these references teach gamma irradiation of nanoparticulate active agent compositions.

C. Background Relating to Sterilization of Nanoparticulate Active Agent Compositions

There are two generally accepted methods for sterilizing pharmaceutical products: heat sterilization and sterile filtration.

1. Heat Sterilization of Nanoparticulate Compositions

One of the problems that may be encountered with heat sterilization of nanoparticulate active agent compositions is the solubilization and subsequent recrystallization of the component active agent particles. This process can result in an increase in the size distribution of the active agent particles. In addition, some nanoparticulate formulations also exhibit particle aggregation following exposure to elevated temperatures during the heat sterilization process.

Crystal growth and particle aggregation in nanoparticulate active agent preparations are highly undesirable for several reasons. The presence of large crystals in the nanoparticulate active agent composition may cause undesirable side effects, especially when the preparation is in an injectable formulation. Larger active agent particles formed by particle aggregation and recrystallization can interfere with blood flow, causing pulmonary embolism and death.

In addition, with both injectable and oral formulations the presence of large active agent crystals, and therefore varying active agent particle sizes, and/or active agent particle aggregation, can change the pharmacokinetic profile of the administered active agent. For oral formulations, the presence of large active agent crystals or aggregates can create a variable bioavailability profile because smaller active agent particles dissolve faster than the larger aggregates or larger crystal particles.

A faster rate of active agent dissolution is generally associated with greater bioavailability and a slower rate of dissolution is generally associated with a lower bioavailability. This is because bioavailability is generally proportional to the surface area of an administered drug and, therefore, bioavailability generally increases with a reduction in the particle size of the dispersed active agent (see U.S. Pat. No. 5,662,833). With a composition having widely varying active agent particle sizes, bioavailability becomes highly variable and inconsistent and dosage determinations become difficult.

Moreover, because such crystal growth and particle aggregation are uncontrollable and unpredictable, the quality of the nanoparticulate active agent composition is inconsistent. For intravenously injected active agent particulate formulations, the presence of large active agent crystals or aggregates can induce an immune systems response which causes the larger active agent particles to be transported by macrophage cells to the liver or spleen and metabolized, in addition to the embolytic effects described above.

Aggregation of nanoparticle active agent compositions upon heating is directly related to the precipitation of the surface stabilizer at temperatures above the cloud point of the surface stabilizer. At this point, the surface stabilizer molecules are likely to dissociate from the nanoparticulate active agent and precipitate, leaving the nanoparticulate active agent unprotected. The unprotected nanoparticulate active agent then aggregates into clusters of active agent particles.

Several methods have been suggested in the prior art for preventing such active agent crystal growth and particle aggregation following heat sterilization, including adding a cloud point modifier or crystal growth modifier to the nanoparticulate active agent composition and purifying the surface stabilizer. For example, U.S. Pat. No. 5,298,262 describes the use of an anionic or cationic cloud point modifier in nanoparticulate active agent compositions, and U.S. Pat. No. 5,346,702 describes nanoparticulate active agent compositions having a nonionic surface stabilizer and a non-ionic cloud point modifier. The cloud point modifier enables heat sterilization of the nanoparticulate active agent compositions with low resultant particle aggregation. U.S. Pat. No. 5,470,583 describes nanoparticulate active agent compositions having a non-ionic surface stabilizer and a charged phospholipid as a cloud point modifier.

The prior art also describes methods of limiting crystal growth in a nanoparticulate active agent composition by adding a crystal growth modifier (see U.S. Pat. Nos. 5,662,883 and 5,665,331). In addition, U.S. Pat. No. 5,302,401 describes nanoparticulate active agent compositions having polyvinylpyrrolidone (PVP) as a surface stabilizer and sucrose as a cryoprotectant (allowing the nanoparticles to be lyophilized). The compositions exhibit minimal active agent particle aggregation following lyophilization.

All of these various prior art methods share one common feature: they require an additional substance added to the nanoparticulate active agent formulation to inhibit or prevent crystal growth and particle aggregation of the nanoparticulate active agent composition. The addition of such a substance can be detrimental as it may induce adverse effects, particularly for injectable formulations. Thus, this minimizes the usefulness of such substances in pharmaceutical compositions. In addition, the requirement of an additional substance to obtain a stable composition increases production costs.

Another method of limiting particle aggregation or crystal growth of nanoparticulate active agent compositions during sterilization known prior to the present invention was the use of purified surface stabilizers. U.S. Pat. No. 5,352,459 describes nanoparticulate active agent compositions having a purified surface stabilizer (having less than 15% impurities) and a cloud point modifier. Purification of surface stabilizers can be expensive and time consuming, thus significantly raising production costs.

2. Sterile Filtration

Filtration is an effective method for sterilizing homogeneous solutions when the membrane filter pore size is less than or equal to about 0.2 microns (200 nm) because a 0.2 micron filter is sufficient to remove essentially all bacteria. Sterile filtration is normally not used to sterilize conventional suspensions of micron-sized active agent particles because the active agent particles are too large to pass through the membrane pores. In principle, 0.2 μm filtration can be used to sterilize nanoparticulate active agent compositions. However, because nanoparticulate active agent compositions have a size range, some of the particles of a typical nanoparticulate active agent composition having an average particle size of 200 nm may have a size greater than 200 nm. Such larger particles tend to clog the sterile filter. Thus, only nanoparticulate active agent compositions having very small average particle sizes can be sterile filtered.

3. Ethylene Oxide Method

The ethylene oxide method has been a widely used sterilization method for suspension/dispersion products where product or components are thermolabile. Most of the currently marketed products utilize this technique by which individual components are sterilized using this method and then processed or assembled together aseptically. The technique, however, requires the elimination of residual ethylene oxide from the product, which is a time consuming and difficult process with the possibility of residual ethylene oxide contaminating the final drug product.

There remains a need in the art for additional methods of sterilizing solid nanoparticulate active agent compositions. The present invention satisfies this need.

### SUMMARY OF THE INVENTION

The present invention is directed to the surprising discovery that solid forms of nanoparticulate active agent compositions can be successfully terminally sterilized via gamma irradiation. The solid that is sterilized by the method of this invention can be formulated into any suitable dosage form. Upon administration or reconstitution in a liquid media, the sterilized solid redisperses into a particle size which is substantially similar to the original nanoparticulate active agent particle size prior to incorporation into a solid.

One aspect of the invention is directed to methods of sterilizing solid forms of nanoparticulate active agent compositions via gamma irradiation. Such a method comprises exposing a solid form of a nanoparticulate active agent composition to a suitable dosage of gamma irradiation. The length of time of irradiation or the total dose of irradiation delivered will depend on the bioburden of the product, the nature of the contaminant, the nature of the product, and the nature of the solid form. The method does not degrade the nanoparticulate active agent or alter the nanoparticulate active agent particle size, and produces a safe and sterile product in compliance with cGMP requirements.

The method according to the present invention can be carried out at ambient temperature and does not require the heating, freezing, filtration, or chemical treatment of the product before the process is carried out. This offers another significant advantage of the present process as it avoids some of the extra treatment steps of the prior art processes.

Another aspect of the invention is directed to solid nanoparticulate active agent compositions sterilized via gamma irradiation. Such compositions comprise at least one active agent and one or more surface stabilizers associated with or adsorbed to the surface of the active agent. The active agent has an effective average particle size of less than about 2 microns. Also encompassed by the invention is a liquid composition comprising a reconstituted solid nanoparticulate active agent compositions sterilized via gamma irradiation.

The present invention is further directed to solid pharmaceutical compositions comprising a sterilized nanoparticulate active agent composition of the invention. The pharmaceutical compositions preferably comprise at least one pharmaceutically acceptable carrier as well as any desired excipients. Also encompassed by the invention is a liquid pharmaceutical composition comprising a reconstituted solid nanoparticulate active agent compositions sterilized via gamma irradiation.

Yet another aspect of the invention encompasses a method of treating a mammal in need comprising administering a therapeutically effective amount of a solid sterilized nanoparticulate active agent composition according to the invention. Also encompassed by the invention is a method of treating a mammal in need comprising administering a therapeutically effective amount of a liquid composition comprising a reconstituted solid nanoparticulate active agent compositions sterilized via gamma irradiation.

Both the foregoing general description and the following detailed description are exemplary and explanatory and are intended to provide further explanation of the invention as claimed. Other objects, advantages, and novel features will be readily apparent to those skilled in the art from the following detailed description of the invention.

### DETAILED DESCRIPTION OF THE INVENTION

The present invention is directed to the surprising and unexpected discovery of a new method for the terminal sterilization of solid nanoparticulate active agent compositions. Nanoparticulate active agent compositions prepared according to methods known in the art are formulated into a solid form, followed by irradiating the solid form with gamma radiation for a period of time sufficient to terminally sterilize the active agent nanoparticles. A solid intermediate can be gamma irradiated, or a final solid dosage form can be gamma irradiated according to the invention.

Suitable solid forms useful for gamma irradiation according to the invention include, but are not limited to, tablets, capsules, dragees, trochees, sachets, lozenges, powders, pills, or granules. Examples of powders include, but are not limited to, lyophilized powders, spray dried powders, spray granulates, etc. The solid form can be, for example, a fast melt dosage form, controlled release dosage form, aerosol dosage form, lyophilized dosage form, delayed release dosage form, extended release dosage form, pulsatile release dosage form, mixed immediate release and controlled release dosage form, or a combination thereof. In addition, the solid form can be formulated for administration via, for example, oral, parenteral, pulmonary, nasal, rectal, local, buccal, ocular, via the ear, or topical administration.

The gamma irradiated solid nanoparticulate active agent can be reconstituted in a liquid, such as water, and used for dosage forms which can be conducive to contamination, such as injectable, aerosol (pulmonary or nasal), or ocular dosage forms, or liquid dosage forms for administration to the ear.

Surprisingly, following sterilization the solid nanoparticulate active agent exhibits unexpected overall stability, maintaining the pre-sterilized physical and chemical properties while meeting cGMP requirements for sterility. The overall stability of the gamma irradiated solid nanoparticulate active agent composition was measured in terms of average particle size, pH, osmolality, percent label claim, content of degradation products, and concentration and molecular weight of the surface stabilizer. (“Label claim” is a measure of what is left of the active ingredient compared to the initial theoretical value (claimed on the package label) after the product has undergone processing or has been stored for some time. It is expressed as a percent of the theoretical value. Thus, preferably values are 100%, or within a range of ˜95% to ˜105% of initial values, and ˜90% to ˜105% for stability.)

It is particularly unexpected that gamma radiation of the solid nanoparticulate active agent does not alter the ability of the solid to redisperse into the component nanoparticles. This is significant as if the sterilized solid formed aggregates or large crystals upon redispersion, the solid would lose the benefits afforded by being formulated into a nanoparticulate active agent composition.

In another aspect of the invention, sterilized solid macroparticulate active agent particles can be combined with the sterilized solid nanoparticulate active agent particles to provide for a sustained or controlled release composition. The combination of very small active agent particles, i.e., nanoparticulate active agent particles, in combination with larger active agent particles, i.e., micronized active agent particles, can enable obtaining the simultaneous presentation of immediate-release (IR) and controlled-release (CR) active agent components. For the purposes of this invention, “nanoparticulate” active agents have an effective average particle size of less than about 2 microns and micronized active agents have an effective average particle size of greater than about 2 microns. The micronized active agent particles can be sterilized via gamma irradiation simultaneously with the nanoparticulate active agent particles or in a separate process.

The nanoparticulate active agent particles, representing the IR component, afford rapid in vivo dissolution, owing to their small size and attendant large specific surface. Alternatively, micronized active agent particles, representing the CR component, afford slower in vivo dissolution, owing to a comparatively large particle size and small attendant specific surface.

IR and CR components representing a wide range of in vivo dissolution rates (and hence, in vivo input rates for absorption) can be engineered through precise control of active agent particle size. Thus, the compositions can comprise a mixture of nanoparticulate active agent particles, wherein each population of particles has a defined size correlating with a precise release rate, and the compositions can comprise a mixture of microparticulate active agent particles, wherein each population of particles has a defined size correlating with a precise release rate.

The present invention is described herein using several definitions, as set forth below and throughout the application.

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

“Conventional active agents or drugs” refers to non-nanoparticulate or solubilized active agents or drugs. Non-nanoparticulate active agents have an effective average particle size of greater than about 2 microns.

The term “microbial” with respect to contamination, as used herein is deemed to include all biological contaminants including bacteria, yeast, and molds.

“Poorly soluble active agents” as used herein means those having a solubility in a liquid media of less than about 30 mg/ml, preferably less than about 20 mg/ml, preferably less than about 10 mg/ml, or preferably less than about 1 mg/ml. Poorly water soluble active agents tend to be eliminated from the gastrointestinal tract before being absorbed into the circulation. Moreover, poorly water soluble active agents tend to be unsafe for intravenous administration techniques, which are used primarily in conjunction with highly water soluble active agents.

As used herein with reference to stable active agent particles, “stable” includes, but is not limited to, one or more of the following parameters: (1) the active agent particles are substantially chemically stable, as measured by degradent concentrations; (2) the active agent particles do not appreciably flocculate or agglomerate due to interparticle attractive forces or otherwise increase in particle size over time; (3) the physical structure of the active agent particles is not altered over time, such as by conversion from an amorphous phase to crystalline phase; (4) where the active agent has not been subjected to a heating step at or above the melting point of the active agent in the preparation of the nanoparticles of the invention.

The term “sterilize” as used in the present application generally means to inactivate substantially all biological contaminants present in the product. In normal pharmaceutical applications, the term “sterilize” is defined as a 6-log (1 million-fold) reduction in the bioburden.

“Therapeutically effective amount” as used herein with respect to an active agent dosage, shall mean that dosage that provides the specific pharmacological response for which the active agent is administered in a significant number of subjects in need of such treatment. It is emphasized that ‘therapeutically effective amount,’ administered to a particular subject in a particular instance will not always be effective in treating the diseases described herein, even though such dosage is deemed a ‘therapeutically effective amount’ by those skilled in the art. It is to be further understood that active agent dosages are, in particular instances, measured as oral dosages, or with reference to drug levels as measured in blood.

A. Redispersibility Profiles of the Sterilized Solid Nanoparticulate Active Agent Compositions of the Invention

The gamma radiation-sterilized solid nanoparticulate active agent compositions of the invention preferably redisperse such that the effective average particle size of the redispersed active agent particles is less than about 2 microns. This is significant, as if upon administration the nanoparticulate active agent compositions of the invention did not redisperse to a substantially nanoparticulate particle size, then the dosage form may lose the benefits afforded by formulating the active agent into a nanoparticulate particle size.

This is because nanoparticulate active agent compositions benefit from the small particle size of the active agent; if the active agent does not redisperse into the small particle sizes upon administration, then “clumps” or agglomerated active agent particles are formed, owing to the extremely high surface free energy of the nanoparticulate active agent system and the thermodynamic driving force to achieve an overall reduction in free energy. With the formation of such agglomerated particles, the bioavailability of the dosage form may fall well below that observed with a form of the nanoparticulate active agent that does not form such agglomerated particles.

Moreover, the gamma radiation-sterilized solid nanoparticulate active agent compositions of the invention preferably exhibit dramatic redispersion of the nanoparticulate active agent particles upon administration to a mammal, such as a human or animal, as demonstrated by reconstitution/redispersion in a biorelevant aqueous media such that the effective average particle size of the redispersed active agent particles is less than about 2 microns. Such biorelevant aqueous media can be any aqueous media that exhibit the desired ionic strength and pH, which form the basis for the biorelevance of the media. The desired pH and ionic strength are those that are representative of physiological conditions found in the human body. Such biorelevant aqueous media can be, for example, aqueous electrolyte solutions or aqueous solutions of any salt, acid, or base, or a combination thereof, which exhibit the desired pH and ionic strength.

Biorelevant pH is well known in the art. For example, in the stomach, the pH ranges from slightly less than 2 (but typically greater than 1) up to 4 or 5. In the small intestine the pH can range from 4 to 6, and in the colon it can range from 6 to 8. Biorelevant ionic strength is also well known in the art. Fasted state gastric fluid has an ionic strength of about 0.1 M while fasted state intestinal fluid has an ionic strength of about 0.14. See e.g., Lindahl et al., “Characterization of Fluids from the Stomach and Proximal Jejunum in Men and Women,” Pharm. Res., 14 (4): 497-502 (1997).

It is believed that the pH and ionic strength of the test solution is more critical than the specific chemical content. Accordingly, appropriate pH and ionic strength values can be obtained through numerous combinations of strong acids, strong bases, salts, single or multiple conjugate acid-base pairs (i.e., weak acids and corresponding salts of that acid), monoprotic and polyprotic electrolytes, etc.

Representative electrolyte solutions can be, but are not limited to, HCl solutions, ranging in concentration from about 0.001 to about 0.1 M, and NaCl solutions, ranging in concentration from about 0.001 to about 0.1 M, and mixtures thereof. For example, electrolyte solutions can be, but are not limited to, about 0.1 M HCl or less, about 0.01 M HCl or less, about 0.001 M HCl or less, about 0.1 M NaCl or less, about 0.01 M NaCl or less, about 0.001 M NaCl or less, and mixtures thereof. Of these electrolyte solutions, 0.01 M HCl and/or 0.1 M NaCl, are most representative of fasted human physiological conditions, owing to the pH and ionic strength conditions of the proximal gastrointestinal tract.

Electrolyte concentrations of 0.001 M HCl, 0.01 M HCl, and 0.1 M HCl correspond to pH 3, pH 2, and pH 1, respectively. Thus, a 0.01 M HCl solution simulates typical acidic conditions found in the stomach. A solution of 0.1 M NaCl provides a reasonable approximation of the ionic strength conditions found throughout the body, including the gastrointestinal fluids, although concentrations higher than 0.1 M may be employed to simulate fed conditions within the human GI tract.

Exemplary solutions of salts, acids, bases or combinations thereof, which exhibit the desired pH and ionic strength, include but are not limited to phosphoric acid/phosphate salts+sodium, potassium and calcium salts of chloride, acetic acid/acetate salts+sodium, potassium and calcium salts of chloride, carbonic acid/bicarbonate salts+sodium, potassium and calcium salts of chloride, and citric acid/citrate salts+sodium, potassium and calcium salts of chloride.

In other embodiments of the invention, the redispersed active agent particles of the invention (redispersed in an aqueous, biorelevant, or any other suitable media) have an effective average particle size of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods.

By “an effective average particle size of less than about 2000 nm” it is meant that at least 50% of the redispersed active agent particles have a particle size of less than the effective average, by weight, i.e., less than about 2000 nm, 1900 nm, 1800 nm, etc. Preferably, at least about 70%, about 90%, about 95%, or about 99% of the redispersed active agent particles have a particle size of less than the effective average, i.e., less than about 2000 nm, 1900 nm, 1800 nm, 1700 nm, etc.

Redispersibility can be tested using any suitable means known in the art. See e.g., the example sections of U.S. Pat. No. 6,375,986 for “Solid Dose Nanoparticulate Compositions Comprising a Synergistic Combination of a Polymeric Surface Stabilizer and Dioctyl Sodium Sulfosuccinate.” Exemplary redispersion media includes, but is not limited to, sterile water for injection, saline, dextrose, Lactated Ringer's solution, and Ringers solution.

B. Active Agents

The active agent may be present either substantially in the form of one optically pure enantiomer or as a mixture, racemic or otherwise, of enantiomers. In addition, the solid active agent exists as a discrete, crystalline phase, as an amorphous phase, a semi-crystalline phase, a semi-amorphous phase, or a combination thereof.

The nanoparticulate active agent particles present in the compositions of the invention have an effective average particle size of less than about 2 microns and are poorly soluble and dispersible in at least one liquid media. The liquid media is preferably water, but can also be, for example, aqueous salt solutions, safflower oil, or a solvent such as ethanol, t-butanol, hexane, or glycol.

Exemplary active agents can be therapeutic or diagnostic agents, collectively referred to as “drugs”. A therapeutic agent can be a pharmaceutical agent, including biologics such as proteins, peptides, and nucleotides, or a diagnostic agent, such as a contrast agent, including x-ray contrast agents.

An active agent can be a pharmaceutical or a diagnostic agent such as a contrast agent or any other type of diagnostic material. The therapeutic or diagnostic agent exists as a crystalline phase, a semi-crystalline phase, an amorphous phase, a semi-amorphous phase, or a mixture thereof.

The active agent can be selected from a variety of known classes of drugs, including, for example, proteins, peptides, NSAIDS, COX-2 inhibitors, nutraceuticals, corticosteroids, elastase inhibitors, analgesics, anti-fungals, oncology therapies, anti-emetics, analgesics, cardiovascular agents, anti-inflammatory agents, anthelmintics, anti-arrhythmic agents, antibiotics (including penicillins), anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytics, sedatives (hypnotics and neuroleptics), astringents, beta-adrenoceptor blocking agents, blood products and substitutes, cardiac inotropic agents, contrast media, corticosteroids, cough suppressants (expectorants and mucolytics), diagnostic agents, diagnostic imaging agents, diuretics, dopaminergics (antiparkinsonian agents), haemostatics, immunological agents, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasodilators, and xanthines.

Examples of representative active agents useful in this invention include, but are not limited to, acyclovir, alprazolam, altretamine, amiloride, amiodarone, benztropine mesylate, bupropion, cabergoline, candesartan, cerivastatin, chlorpromazine, ciprofloxacin, cisapride, clarithromycin, clonidine, clopidogrel, cyclobenzaprine, cyproheptadine, delavirdine, desmopressin, diltiazem, dipyridamole, dolasetron, enalapril maleate, enalaprilat, famotidine, felodipine, furazolidone, glipizide, irbesartan, ketoconazole, lansoprazole, loratadine, loxapine, mebendazole, mercaptopurine, milrinone lactate, minocycline, mitoxantrone, nelfinavir mesylate, nimodipine, norfloxacin, olanzapine, omeprazole, penciclovir, pimozide, tacolimus, quazepam, raloxifene, rifabutin, rifampin, risperidone, rizatriptan, saquinavir, sertraline, sildenafil, acetyl-sulfisoxazole, temazepam, thiabendazole, thioguanine, trandolapril, triamterene, trimetrexate, troglitazone, trovafloxacin, verapamil, vinblastine sulfate, mycophenolate, atovaquone, atovaquone, proguanil, ceftazidime, cefuroxime, etoposide, terbinafine, thalidomide, fluconazole, amsacrine, dacarbazine, teniposide, and acetylsalicylate.

Exemplary nutraceuticals and dietary supplements are disclosed, for example, in Roberts et al., Nutraceuticals: The Complete Encyclopedia of Supplements, Herbs, Vitamins, and Healing Foods (American Nutraceutical Association, 2001), which is specifically incorporated by reference. A nutraceutical or dietary supplement, also known as a phytochemical or functional food, is generally any one of a class of dietary supplements, vitamins, minerals, herbs, or healing foods that have medical or pharmaceutical effects on the body. Exemplary nutraceuticals or dietary supplements include, but are not limited to, folic acid, fatty acids (e.g., DHA and ARA), fruit and vegetable extracts, vitamins, minerals, phosphatidylserine, lipoic acid, melatonin, glucosamine/chondroitin, Aloe Vera, Guggul, glutamine, amino acids (e.g., iso-leucine, leucine, lysine, methionine, phenylanine, threonine, tryptophan, and valine), green tea, lycopene, whole foods, food additives, herbs, phytonutrients, antioxidants, flavonoid constituents of fruits, evening primrose oil, flax seeds, fish and marine animal oils, and probiotics. Nutraceuticals and dietary supplements also include bio-engineered foods genetically engineered to have a desired property, also known as “pharmafoods.”

A description of these classes of active agents and a listing of species within each class can be found in Martindale, The Extra Pharmacopoeia, 31ₛₜ Edition (The Pharmaceutical Press, London, 1996), specifically incorporated herein by reference. The active agents are commercially available and/or can be prepared by techniques known in the art.

C. Surface Stabilizers for Nanoparticulate Active Agents

If the active agent has a nanoparticulate particle size prior to incorporation into a solid form, with “nanoparticulate” being defined as an effective average particle size of less than about 2 microns, then the active agent generally will have at least one surface stabilizer associated with or adsorbed on the surface of the active agent.

Surface stabilizers useful herein physically adhere on or associate with the surface of the nanoparticulate active agent but do not chemically react with the active agent particles. Individual molecules of the surface stabilizer are preferably essentially free of intermolecular cross-linkages.

Exemplary useful surface stabilizers include, but are not limited to, known organic and inorganic pharmaceutical excipients. Such excipients include various polymers, low molecular weight oligomers, natural products, and surfactants. Useful surface stabilizers include nonionic surface stabilizers, ionic surface stabilizers, cationic surface stabilizers, and zwitterionic surface stabilizers. Combinations of more than one surface stabilizer can be used in the invention.

Representative examples of surface stabilizers include hydroxypropyl methylcellulose, hydroxypropylcellulose, polyvinylpyrrolidone (PVP), random copolymers of vinyl pyrrolidone and vinyl acetate, sodium lauryl sulfate, dioctylsulfosuccinate, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween 20® and Tween 80® (ICI Speciality Chemicals)); polyethylene glycols (e.g., Carbowaxs 3550® and 934® (Union Carbide)), polyoxyethylene stearates, colloidal silicon dioxide, phosphates, carboxymethylcellulose calcium, carboxymethylcellulose sodium, methylcellulose, hydroxyethylcellulose, hydroxypropylmethylcellulose phthalate, noncrystalline cellulose, magnesium aluminium silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics F68® and F108®, which are block copolymers of ethylene oxide and propylene oxide); poloxamines (e.g., Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.); Tetronic 1508® (T-1508) (BASF Wyandotte Corporation), Tritons X-200®, which is an alkyl aryl polyether sulfonate ( Dow); Crodestas F-110®, which is a mixture of sucrose stearate and sucrose distearate (Croda Inc.); p-isononylphenoxypoly-(glycidol), also known as Olin-lOG® or Surfactant 10-G® (Olin Chemicals, Stamford, Conn.); Crodestas SL-40® (Croda, Inc.); and SA9OHCO, which is C₁₈H₃₇CH₂C(O)N(CH₃)—CH₂(CHOH)₄(CH₂0H)₂ (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; PEG-phospholipid, PEG-cholesterol, PEG-cholesterol derivative, PEG-vitamin A, PEG-vitamin E, lysozyme, and the like.

Examples of useful cationic surface stabilizers include, but are not limited to, polymers, biopolymers, polysaccharides, cellulosics, alginates, phospholipids, and nonpolymeric compounds, such as zwitterionic stabilizers, poly-n-methylpyridinium, anthryul pyridinium chloride, cationic phospholipids, chitosan, polylysine, polyvinylimidazole, polybrene, polymethylmethacrylate trimethylammoniumbromide bromide (PMMTMABr), hexyldesyltrimethylammonium bromide (HDMAB), and polyvinylpyrrolidone-2-dimethylaminoethyl methacrylate dimethyl sulfate.

Other useful cationic stabilizers include, but are not limited to, cationic lipids, sulfonium, phosphonium, and quarternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C₁₂₋₁₅dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl (ethenoxy)₄ ammonium chloride or bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂, C₁₅, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336™), POLYQUAT 10™, tetrabutylammonium bromide, benzyl trimethylammonium bromide, choline esters (such as choline esters of fatty acids), benzalkonium chloride, stearalkonium chloride compounds (such as stearyltrimonium chloride and Di-stearyldimonium chloride), cetyl pyridinium bromide or chloride, halide salts of quatemized polyoxyethylalkylamines, MIRAPOL™ and ALKAQUAT™ (Alkaril Chemical Company), alkyl pyridinium salts; amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides; imide azolinium salts; protonated quaternary acrylamides; methylated quaternary polymers, such as poly[diallyl dimethylammonium chloride] and poly-[N-methyl vinyl pyridinium chloride]; and cationic guar.

Such exemplary cationic surface stabilizers and other useful cationic surface stabilizers are described in J. Cross and E. Singer, Cationic Surfactants: Analytical and Biological Evaluation (Marcel Dekker, 1994); P. and D. Rubingh (Editor), Cationic Surfactants: Physical Chemistry (Marcel Dekker, 1991); and J. Richmond, Cationic Surfactants: Organic Chemistry, (Marcel Dekker, 1990).

Particularly preferred nonpolymeric primary stabilizers are any nonpolymeric compound, such benzalkonium chloride, a carbonium compound, a phosphonium compound, an oxonium compound, a halonium compound, a cationic organometallic compound, a quarternary phosphorous compound, a pyridinium compound, an anilinium compound, an ammonium compound, a hydroxylammonium compound, a primary ammonium compound, a secondary ammonium compound, a tertiary ammonium compound, and quarternary ammonium compounds of the formula NR₁R₂R₃R₄⁽⁺⁾. For compounds of the formula NR₁R₂R₃R₄⁽⁺⁾:

(i) none of R₁-R₄ are CH₃;

(ii) one of R₁-R₄ is CH₃;

(iii) three of R₁-R₄ are CH₃;

(iv) all of R₁-R₄ are CH₃;

(v) two of R₁-R₄ are CH₃, one of R₁-R₄ is C₆H₅CH₂, and one of R₁-R₄ is an alkyl chain of seven carbon atoms or less;

(vi) two of R₁-R₄ are CH₃, one of R₁-R₄ is C₆H₅CH₂, and one of R₁-R₄ is an alkyl chain of nineteen carbon atoms or more;

(vii)two of R₁-R₄ are CH₃ and one of R₁-R₄ is the group C₆H₅(CH₂)ₙ, where n>1;

(viii) two of R₁-R₄ are CH₃, one of R₁-R₄ is C₆H₅CH₂, and one of R₁-R₄ comprises at least one heteroatom;

(ix) two of R₁-R₄ are CH₃, one of R₁-R₄ is C₆H₅CH₂, and one of R₁-R₄ comprises at least one halogen;

(x) two of R₁-R₄ are CH₃, one of R₁-R₄ is C₆H₅CH₂, and one of R₁-R₄ comprises at least one cyclic fragment;

(xi) two of R₁-R₄ are CH₃ and one of R₁-R₄ is a phenyl ring; or

(xii) two of R₁-R₄ are CH₃ and two of R₁-R₄ are purely aliphatic fragments.

Such compounds include, but are not limited to, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride(Quatemium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

Most of these surface stabilizers are known pharmaceutical excipients and are described in detail in the Handbook of Phannaceutical Excipients, published jointly by the American Pharmaceutical Association and The Pharmaceutical Society of Great Britain (The Pharmaceutical Press, 2000), specifically incorporated by reference. The surface stabilizers are commercially available and/or can be prepared by techniques known in the art.

D. Particle Size of the Active Agent

As used herein, particle size is determined on the basis of the weight average particle size as measured by conventional particle size measuring techniques well known to those skilled in the art. Such techniques include, for example, sedimentation field flow fractionation, photon correlation spectroscopy, light scattering, and disk centrifugation.

By “an effective average particle size of less than about 2 microns” it is meant that at least 50% of the active agent particles have a size of less than about 2 microns, when measured by the above techniques. In other embodiments of the invention, at least about 70%, at least about 90%, at least about 95%, or at least about 99% of the active agent particles have a particle size less than the effective average, i.e., less than about 2 microns.

In addition, in other embodiments of the invention, the effective average particle size of the nanoparticulate active agent particles can be less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 nm, less than about 75 nm, or less than about 50 nm.

In the present invention, the value for D50 of a nanoparticulate active agent composition is the particle size below which 50% of the active agent particles fall, by weight. Similarly, D90 is the particle size below which 90% of the active agent particles fall, by weight.

For conventional or microparticulate active agents, by “an effective average particle size of greater than about 2 microns” it is meant that at least 50% of the active agent particles have a particle size greater than about 2 microns, when measured by the above techniques. In other embodiments of the invention, at least about 70%, at least about 90%, at least about 95%, or at least about 99% of the active agent particles have a size greater than about 2 microns, when measured by the above techniques.

E. Concentration of Nanoparticulate Active Agent and Surface Stabilizer

If the active agent is in a nanoparticulate particle size, then the active agent has one or more surface stabilizers adsorbed on or associated with the surface of the agent. The relative amount of active agent and one or more surface stabilizers can vary widely. The optimal amount of the surface stabilizer(s) can depend, for example, upon the particular active agent selected, the equivalent hydrophilic lipophilic balance (HLB) of the active agent, the melting point, cloud point, and water solubility of the surface stabilizer, and the surface tension of water solutions of the stabilizer, etc.

The concentration of at least one active agent can vary from about 99.5% to about 0.001%, from about 95% to about 0.1%, or from about 90% to about 0.5%, by weight, based on the total combined weight of the at least one active agent and at least one surface stabilizer, not including other excipients.

The concentration of at least one surface stabilizer can vary from about 0.5% to about 99.999%, from about 5% to about 99.9%, and from about 10% to about 99.5%, by weight, based on the total combined dry weight of at least one active agent and at least one surface stabilizer, not including other excipients.

F. Other Pharmaceutical Excipients

Pharmaceutical compositions according to the invention may also comprise one or more binding agents, filling agents, lubricating agents, suspending agents, sweeteners, flavoring agents, preservatives, buffers, wetting agents, disintegrants, effervescent agents, and other excipients. Such excipients are known in the art.

Examples of filling agents are lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicifized microcrystalline cellulose (SMCC).

Suitable lubricants, including agents that act on the flowability of a powder to be compressed, are colloidal silicon dioxide, such as Aerosil® 200; talc, stearic acid, magnesium stearate, calcium stearate, and silica gel.

Examples of sweeteners are any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like.

Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quarternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinylpyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the acid component of the effervescent couple may be present.

Compositions suitable for parenteral injection may comprise physiologically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents, or vehicles including water, ethanol, sodium chloride, Ringer's solution, lactated Ringer's solution, stabilizer solutions, tonicity enhancers (sucrose, dextrose, mannitol, etc.) polyols (propyleneglycol, polyethylene-glycol, glycerol, and the like), suitable mixtures thereof, vegetable oils (such as olive oil) and injectable organic esters such as ethyl oleate. Suitable fluids are referenced in Remington's Pharmaceutical Sciences, 17^{th} edition, published by Mack Publishing Co., page 1543.

G. Methods of Making Nanoparticulate Active Agent Compositions

Nanoparticulate active agent compositions can be made using methods known in the art such as, for example, milling, homogenization, and precipitation techniques. Exemplary methods of making nanoparticulate active agent compositions are described in U.S. Pat. No. 5,145,684.

Methods of making nanoparticulate active agent compositions are also described in U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” U.S. Pat. No. 5,665,331, for “Co-Microprecipitation of Nanoparticulate Pharmaceutical Agents with Crystal Growth Modifiers;” U.S. Pat. No. 5,662,883, for “Co-Microprecipitation of Nanoparticulate Pharmaceutical Agents with Crystal Growth Modifiers;” U.S. Pat. No. 5,560,932, for “Microprecipitation of Nanoparticulate Pharmaceutical Agents;” U.S. Pat. No. 5,543,133, for “Process of Preparing X-Ray Contrast Compositions Containing Nanoparticles;” U.S. Pat. No. 5,534,270, for “Method of Preparing Stable Drug Nanoparticles;” U.S. Pat. No. 5,510,118, for “Process of Preparing Therapeutic Compositions Containing Nanoparticles;” and U.S. Pat. No. 5,470,583, for “Method of Preparing Nanoparticle Compositions Containing Charged Phospholipids to Reduce Aggregation,” all of which are specifically incorporated by reference.

1. Milling to Obtain Nanoparticulate Active Agent Dispersions

Milling of aqueous active agent dispersions to obtain a dispersion of a nanoparticulate active agent comprises dispersing at least one active agent in a liquid dispersion media in which the active agent is poorly soluble. By “poorly soluble” it is meant that the active agent has a solubility in the liquid dispersion media of less than about 30 mg/ml, less than about 20 mg/ml, preferably less than about 10 mg/ml, and more preferably less than about 1 mg/ml. Such a liquid dispersion media can be, for example, water, aqueous salt solutions, oils such as safflower oil, and solvents such as ethanol, t-butanol, hexane, and glycol.

This is followed by applying mechanical means in the presence of grinding media to reduce the particle size of the active agent to the desired effective average particle size. The active agent particles can be reduced in size in the presence of at least one surface stabilizer. Alternatively, the active agent particles may be contacted with one or more surface stabilizers after attrition. Other compounds, such as a diluent, can be added to the active agent/surface stabilizer composition during the size reduction process. Dispersions can be manufactured continuously or in a batch mode. The resultant nanoparticulate active agent dispersion can then be formulated into a solid form, followed by gamma irradiation of the solid form.

2. Precipitation to Obtain Nanoparticulate Active Agent Compositions

Another method of forming the desired nanoparticulate active agent composition is by microprecipitation. This is a method of preparing stable dispersions of poorly soluble active agents in the presence of one or more surface stabilizers and one or more colloid stability enhancing surface active agents free of any trace toxic solvents or solubilized heavy metal impurities. Such a method comprises, for example: (1) dissolving the poorly soluble active agent in a suitable solvent; (2) adding the formulation from step (1) to a solution comprising at least one surface stabilizer to form a solution; and (3) precipitating the formulation from step (2) using an appropriate non-solvent. The method can be followed by removal of any formed salt, if present, by dialysis or diafiltration and concentration of the dispersion by conventional means. The resultant nanoparticulate active agent dispersion can then be formulated into a solid form, followed by gamma irradiation of the solid form.

3. Homogenization to Obtain Nanoparticulate Active Agent Compositions

Exemplary homogenization methods of preparing nanoparticulate active agent compositions are described in U.S. Pat. No. 5,510,118, for “Process of Preparing Therapeutic Compositions Containing Nanoparticles.”

Such a method comprises dispersing active agent particles in a liquid dispersion media, followed by subjecting the dispersion to homogenization to reduce the particle size of the active agent to the desired effective average particle size. The active agent particles can be reduced in size in the presence of at least one surface stabilizer. Alternatively, the active agent particles can be contacted with one or more surface stabilizers either before or after particle size reduction. It is preferred, however, to disperse the active agent particles in the liquid dispersion media in the presence of at least one surface stabilizer as an aid to wetting of the active agent particles. Other compounds, such as a diluent, can be added to the active agent/surface stabilizer composition either before, during, or after the particle size reduction process. Dispersions can be manufactured continuously or in a batch mode. The resultant nanoparticulate active agent dispersion can then be formulated into a solid form, followed by gamma irradiation of the solid form.

H. Methods of Making Solid Forms of Nanoparticulate Active Agent Compositions

1. Spray Drying of Nanoparticulate Active Agent Dispersions

Solid forms of nanoparticulate active agent dispersions can be prepared by drying the liquid nanoparticulate active agent dispersion following particle size reduction. A preferred drying method is spray drying.

In an exemplary spray drying process, the nanoparticulate active agent dispersion is fed to an atomizer using a peristaltic pump and atomized into a fine spray of droplets. The spray is contacted with hot air in the drying chamber resulting in the evaporation of moisture from the droplets. The resulting spray is passed into a cyclone where the powder is separated and collected. The nanoparticulate active agent dispersion can be spray-dried in the presence or absence of excipients.

The spray-dried powder can be gamma irradiated, or the powder can be further processed into a solid dosage form such as a tablet, sachet, etc., followed by gamma irradiation of the solid dosage form. Gamma irradiated spray-dried powders of nanoparticulate active agents can also be formulated into an aerosol for nasal or pulmonary administration, or the powder can be redispersed in a liquid dispersion media and the subsequent liquid dosage form can be used in a suitable application, such as in oral compositions, injectable compositions, ocular compositions, liquid nasal and pulmonary aerosols, ear drops, etc.

2. Lyophilization of Nanoparticulate Active Agent Dispersions

Solid or powder forms of nanoparticulate active agent dispersions can also be prepared by lyophilizing the liquid nanoparticulate active agent dispersion following particle size reduction.

In the lyophilization step, water is removed from the nanoparticulate active agent formulations after the dispersion is frozen and placed under vacuum, allowing the ice to change directly from solid to vapor without passing through a liquid phase. The lyophilization process consists of four interdependent processes: freezing, sublimation, the primary drying step, and desorption, which is the secondary drying step. Many lyophilizers can be used to achieve the lyophilization step of nanoparticulate active agent dispersions.

Suitable lyophilization conditions include, for example, those described in EP 0,363,365 (McNeil-PPC Inc.), U.S. Pat. No. 4,178,695 (A. Erbeia), and U.S. Pat. No. 5,384,124 (Farmalyoc), all of which are incorporated herein by reference. Typically, the nanoparticulate active agent dispersion is placed in a suitable vessel and frozen to a temperature of between about −5° C. to about −100° C. The frozen dispersion is then subjected to reduced pressure for a period of up to about 48 hours. The combination of parameters such as temperature, pressure, dispersion media, and batch size will impact the time required for the lyophilization process. Under conditions of reduced temperature and pressure, the frozen solvent is removed by sublimation yielding a solid, porous, immediate release solid dosage form having the nanoparticulate active agent distributed throughout.

Following gamma irradiation, the lyophilized solid form can be formulated, for example, into a powder, tablet, suppository, or other solid dosage form, a powder can be formulated into an aerosol for nasal or pulmonary administration, or a powder can be reconstituted into a liquid dosage form, such as ocular drops, liquid nasal and pulmonary aerosols, ear drops, injectable compositions, etc.

3. Granulation Nanoparticulate Active Agent Dispersions

A solid form of the invention can be prepared by granulating in a fluidized bed an admixture comprising a nanoparticulate active agent dispersion, comprising at least one surface stabilizer, with a solution of at least one pharmaceutically acceptable water-soluble or water-dispersible excipient, to form a granulate. This can be followed by gamma irradiation of the granulate, or gamma irradiation of a solid dosage form prepared from the granulate.

4. Tableting

The solid forms of the invention can be in the form of tablets. Preparation of such tablets can be, for example, by pharmaceutical compression or molding techniques known in the art. The tablets of the invention may take any appropriate shape, such as discoid, round, oval, oblong, cylindrical, triangular, hexagonal, and the like.

Powders for tableting can be formulated into tablets by any method known in the art. Suitable methods include, but are not limited to, milling, fluid bed granulation, dry granulation, direct compression, spheronization, spray congealing, and spray-dying. Detailed descriptions of tableting methods are provided in Remington: The Science and Practice of Pharmacy, 19th ed. Vol. 11 (1995) (Mack Publishing Co., Pennsylvania); and Remington's Pharmaceutical Sciences, Chapter 89, pp. 1633-1658 (Mach Publishing Company, 1990), both of which are specifically incorporated by reference.

The tablets may be coated or uncoated. If coated they may be sugar-coated (to cover objectionable tastes or odors and to protect against oxidation) or film coated (a thin film of water soluble matter for similar purposes).

I. Gamma Radiation

The solid nanoparticulate active agent particles are subjected to gamma radiation at ambient temperature, which remains relatively constant during the period of irradiation. Gamma radiation is applied in an amount sufficient to destroy substantially all of the microbial contamination in the solid form. In addition, the rate of radiation generated in the radiation chamber is relatively constant during the entire radiation period. The total amount of gamma radiation that the solid nanoparticulate active agent is exposed to has been experimentally verified to: (1) render the active agent composition sterile, and (2) maintain the integrity of the nanoparticulate active agent composition. The application of the gamma radiation does not significantly degrade the active agent or reduce the active agent's efficacy. In this way, it is possible to provide products which meet cGMP requirements for sterile products without harming the active agent.

In a preferred aspect of the invention, the gamma radiation is applied in a preferred cumulative amount of about 5 kGray to about 50 kGray or less. Generally, the gamma radiation will normally be applied in a range of about 5 kGray to about 25 kGray or less.

The microbial contamination which is to be destroyed is generally that of bacterial contamination and mycoplasma contamination.

One of the principal aspects of the invention is that upon reconstitution or redispersion after gamma irradiation, the terminally sterilized solid nanoparticulate active agent maintains its overall stability. Specifically the terminally sterilized solid nanoparticulate active agent maintains its redispersibility as evidenced by a retention of particle size, pH, osmolality, assay, and stabilizer concentration following redispersion of the solid n a liquid media, as detailed in the examples that follow.

J. Administration of the Compositions of the Invention

The present invention provides a method of treating a mammal, including a human, requiring administration of a sterile dosage form. As used herein, the term “subject” is used to mean an animal, preferably a mammal, including a human. The terms “patient” and “subject” may be used interchangeably.

Examples of particularly useful applications of such dosage forms include injectable dosage forms, aerosol dosage forms, and dosage forms to be administered to immunocompromised subjects, subjects being treated with immunosuppressants, such as transplant subjects, elderly subjects, and juvenile or infant subjects.

The sterile dosage forms of the invention can be administered to a subject via any conventional method including, but not limited to, orally, rectally, vaginally, ocularly, parenterally (e.g., intravenous, intramuscular, or subcutaneous), intracisternally, pulmonary, intravaginally, intraperitoneally, locally (e.g., ointments or drops), via the ear, or as a buccal or nasal spray.

Sterile dosage forms suitable for parenteral injection may include physiologically acceptable sterile aqueous or nonaqueous solutions, dispersions, suspensions or emulsions, and sterile powders for reconstitution into sterile injectable solutions or dispersions. Proper fluidity can be maintained, for example, by the use of a coating such as lecithin, by the maintenance of the required particle size in the case of dispersions, and by the use of surfactants.

Sterile dosage forms for oral administration may include pharmaceutically acceptable emulsions, solutions, suspensions, syrups, and elixirs. In addition to the active agent and surface stabilizer, the sterile dosage forms may include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propylene glycol, 1,3-butyleneglycol, dimethylformamide, oils, such as cottonseed oil, groundnut oil, corn germ oil, olive oil, castor oil, and sesame oil, glycerol, tetrahydrofurfuryl alcohol, polyethyleneglycols, fatty acid esters of sorbitan, or mixtures of these substances, and the like.

In general, the sterile dosage forms of the invention will be administered to a mammalian subject in need thereof using a level of drug or active agent that is sufficient to provide the desired physiological effect. The effective amounts of the active agent of the composition of the invention can be determined empirically and can be employed in pure form or, where such forms exist, in pharmaceutically acceptable salt, ester, or prodrug form. Actual dosage levels of the active agent in the sterile dosage form of the invention may be varied to obtain an amount of the active agent that is effective to obtain a desired therapeutic response for a particular composition and method of administration and the condition to be treated. The selected dosage level therefore depends upon the desired therapeutic effect, the route of administration, the potency of the administered active agent, the desired duration of treatment, and other factors. The level of active agent needed to give the desired physiological result is readily determined by one of ordinary skill in the art by referring to standard texts, such as Goodman and Gillman and the Physician's Desk Reference.

Dosage unit compositions may contain such amounts of such submultiples thereof as may be used to make up the daily dose. It will be understood, however, that the specific dose level for any particular subject will depend upon a variety of factors: the type and degree of the cellular or physiological response to be achieved; activity of the specific agent or composition employed; the specific agent(s) or composition employed; the age, body weight, general health, sex, and diet of the patient; the time of administration, route of administration, and rate of excretion of the active agent; the duration of the treatment; active agents used in combination or coincidental with the specific active agent; and like factors well known in the medical arts.

The following examples are given to illustrate the present invention. It should be understood, however, that the invention is not to be limited to the specific conditions or details described in these examples. Throughout the specification, any and all references to a publicly available document, including a U.S. patent, are specifically incorporated by reference.

Examples have been set forth below for purposes of illustration and to describe the best mode of the invention at the present time. The scope of the invention is not to be in any way limited by the examples set forth herein.

### EXAMPLE 1

The purpose of this example was to prepare two liquid nanoparticulate naproxen formulations which exhibited good overall stability, formulation of a solid form of the two liquid nanoparticulate naproxen formulations via lyophilization, followed by terminal sterilization of the two solid lyophilized formulations by gamma irradiation.

Formulation 1 comprised 20% (w/w) naproxen, 2% (w/w) polyvinylpyrrolidone (PVP), and sodium hydroxide (NaOH), and Formulation 2 comprised 20% (w/w) naproxen, 2% (w/w) PVP, and 4% (w/w) histidine.

A. Preparation of Solid Naproxen Formulation 1

A 2L stainless steel recirculation vessel was cleaned with 70% isopropyl alcohol and dried. 30 g of PVP (Kollidon® 12 PF, BASF) was stirred into 1170 g of sterile water for injection (Abbott Laboratories) in the 2L stainless steel recirculation vessel until the PVP dissolved. 300 g of naproxen (Alfa Chemical) was stirred into the resulting solution until the naproxen was thoroughly wetted. The pH of the resulting slurry was adjusted to approximately 7 by adding dropwise a 50% (w/w) NaOH solution.

Subsequently, PolyMill™-200 (DOW) milling media was charged via a vacuum into a 600 cc continuous feed milling chamber of a DYNO®-Mill (Type: KDL, manufactured by Willy Bachofen, AG Maschinenfabrik). The naproxen/PVP slurry was milled for approximately 2.5 hours. An autoclaved filter apparatus and a 100 μm mesh screen were used in a laminar flow hood to harvest a nanoparticulate naproxen dispersion. The bulk dispersion was filtered through a 5 μm PolyCap™ 36 HD (Whatman) filter into an autoclaved receiver vessel in a laminar flow hood. 1281.72 g of nanoparticulate naproxen dispersion was collected having a mean particle size of 125 nm.

Approximately 4 g of nanoparticulate naproxen dispersion (Formulation 1) was dispensed into 10 mL autoclaved glass vials, which were stoppered with autoclaved 20 mm gray butyl (Kimble) stoppers.

The stoppered samples had their stoppers loosened so that air could enter and were then transferred to a Dura-Stop™ Lyophilizer (FTS™ Systems), where the samples were lyophilized. Prior to removing the samples from the lyophilizer, they were stoppered under vacuum. The samples were then removed from the lyophilizer and crimped for the sterilization step.

Thereafter, the lyophilized samples were terminally sterilized via gamma irradiation at a dose of 25 kGray.

B. Preparation of Solid Naproxen Formulation 2

A 2L stainless steel recirculation vessel was cleaned with 70% isopropyl alcohol and dried. 30 g of PVP (Kollidon® 12 PF, BASF) was stirred into 1170 g of sterile water for injection (Abbott Laboratories) in the 2 L stainless steel recirculation vessel until the PVP dissolved. 60 g of L-Histidine (Sigma) was stirred into the resulting solution. 300 g of naproxen (Alfa Chemical) was stirred into the resulting PVP/L-Histidine solution until the naproxen was thoroughly wetted. The pH of the resulting naproxen slurry was approximately 6.9.

Subsequently, PolyMill™-200 (DOW) milling media was charged via a vacuum into a 600 cc continuous feed milling chamber of a DYNO®-Mill (Type: KDL, manufactured by Willy Bachofen, AG Maschinenfabrik), and the naproxen containing slurry was milled for approximately 3 hours. An autoclaved filter apparatus and a 100 μm mesh screen were used in a laminar flowhood to harvest the nanoparticulate naproxen dispersion. The bulk dispersion was filtered with a 5 μm PolyCap™ 36 HD (Whatman) filter into an autoclaved receiver vessel in a laminar flow hood. 1164 g of the nanoparticulate naproxen dispersion was collected having a mean particle size of 175 nm.

Approximately 4 g of the nanoparticulate naproxen dispersion (Formulation 2) was dispensed into 10 mL autoclaved glass vials which were stoppered with autoclaved 20 mm gray butyl (Kimble) stoppers. The stoppered samples had their stoppers loosened so that air could enter and were then transferred to a Dura-Stop™ Lyophilizer (FTS™ Systems) where the samples were lyophilized. Prior to removing the samples from the lyophilizer, they were stoppered under vacuum. The samples were then removed from the lyophilizer and crimped for the sterilization step.

Thereafter, the lyophilized samples were terminally sterilized via gamma irradiation at a dose of 25 kGray.

### Example 2

Analysis of Formulations 1 and 2

In this example, samples of pre-lyophilized liquid nanoparticulate naproxen dispersions, solid lyophilized (LYO) nanoparticulate naproxen dispersions, and gamma irradiated solid lyophilized (GIL) nanoparticulate naproxen dispersions were tested for certain physico-chemical properties to ascertain that the pharmaceutical formulations sterilized according to the invention complied with current good manufacturing practice (cGMP) requirements for sterility of parenteral products.

Liquid Formulations 1 and 2, prepared as in Example 1 above, were tested for particle size, optical microscopy, pH, osmolality, naproxen assay, and concentration of PVP to ascertain that the formulation degradation profile is acceptable. Upon observation by optical microscopy, both of the formulations were homogeneous and free-flowing dispersions.

A. Particle Size Analysis

Pre-lyophilization, post-lyophilization, and post-gamma irradiation samples of Formulations 1 and 2 were analyzed for particle size using a Horiba LA-910 Static Light Scattering Particle Analyzer (Horiba Instruments, Irvine, Calif.). Each sample was measured without sonication and following one minute of sonication to determine whether the dispersions were aggregated (if aggregation was present in the sample, then the particle size of the sample would be significantly smaller following sonication). The reported values from the histograms included Dₘₑₐₙ and D₉₀ particle size. The results for samples of Formulations 1 and 2 are set forth in Tables 1 and 2 below.

B. pH Measurement

The pH values of all of the naproxen samples (pre-lyophilization, post-lyophilization, and post-gamma irradiation) were measured using a Beckman Φ 720 pH meter. The pH meter was calibrated prior to use with pH=4 and pH=7 calibration buffer solutions. The pH results for all samples are set forth below in Tables 1 and 2.

C. Osmolality Measurements

The osmolality values of the naproxen post-lyophilization and post-gamma irradiation samples were measured using a Wescor Vapro® Vapor Pressure Osmometer. A 10 μL sample of reconstituted nanoparticulate naproxen dispersion was used for each measurement. The osmometer was calibrated prior to use with a 290 mmol/kg standard solution. The osmolality results obtained for samples of Formulations 1 and 2 are set forth in Tables 1 and 2 below.

TABLE 1
Formulation 1: Particle Size, pH, and Osmolality Stability

Type

| Formulation | | DMean (nm) | | D90 (nm) | | | Osmolality |
|---|---|---|---|---|---|---|---|
| 1** | Time | no son.* | 1 min. son. | No son. | 1 min. son. | pH | mOsm/kg |
| NCD*** | Initial | 125 | 125 | 164 | 165 | 7.0 | — |
| | 24 hr@5° C. | 123 | 124 | 163 | 163 | — | — |
| LYO | Initial | 131 | 131 | 174 | 174 | 6.8 | 90 |
| | 3 mo. | 132 | 135 | 177 | 183 | 6.9 | 89 |
| | 6 mo. | 142 | 144 | 190 | 193 | 6.9 | 80 |
| GIL | Initial | 132 | 132 | 176 | 175 | 6.8 | 89 |
| | 3 mo. | 135 | 135 | 183 | 183 | 6.9 | 93 |
| | 6 mo. | 146 | 143 | 196 | 191 | 6.9 | 81 |

*no son. - without sonication
**20% Naproxen + 2% PVP + NaOH (Formulation 1)
NCD - Pre-lyophilized nanoparticulate naproxen dispersion
LYO Lyophilized nanoparticulate naproxen dispersion
GIL—Gamma Irradiated Lyophilized nanoparticulate naproxen dispersion
***All samples were stored at room temperature except for the NCD, which was stored at 5° C.

TABLE 2
Formulation 2: Particle Size, pH and Osmolality Stability

| Type | | Particle Size | | Particle Size | | | |
|---|---|---|---|---|---|---|---|
| Formulation | | DMean (nm) | | D90 (nm) | | | Osmolality |
| 2** | Time | no son.* | 1 min. son. | No son. | 1 min. son. | pH | MOsm/kg |
| NCD*** | Initial | 175 | 177 | 232 | 235 | 6.8 | — |
| | 24 hr@5° C. | 186 | 189 | 248 | 251 | — | — |
| LYO | Initial | 239 | 269 | 339 | 397 | 6.8 | 346 |
| | 3 mo. | 247 | 231 | 322 | 300 | 6.9 | 359 |
| | 6 mo. | 225 | 225 | 290 | 290 | 6.9 | 361 |
| GIL | Initial | 224 | 224 | 292 | 292 | 6.8 | 354 |
| | 3 mo. | 202 | 203 | 268 | 269 | 6.9 | 356 |
| | 6 mo. | 235 | 228 | 301 | 292 | 6.9 | 367 |

*no son. - without sonication
**20% Naproxen + 2% PVP + 4% L-Histidine (Formulation 2)
NCD - Pre-lyophilized nanoparticulate naproxen dispersion
LYO - Lyophilized nanoparticulate naproxen dispersion
GIL—Gamma Irradiated Lyophilized nanoparticulate naproxen dispersion
***All samples were stored at room temperature except for the NCD, which was stored at 5° C.

D. Particle Size Results

From Table 1 above, it is surprisingly apparent that the initial mean particle size of the lyophilized product of Formulation 1 remained unchanged, with values of 131 nm and 132 nm for pre- and post-gamma irradiated samples, respectively. These unexpected findings also compared to the initial nanoparticulate naproxen dispersion (NCD) particle size of 125 nm, indicating that hardly any agglomeration of particles occurred in the post-gamma irradiated samples.

After 3 months of storage at ambient temperature, the particle size of the LYO and GIL samples remained essentially unchanged, with a mean particle size of 132 nm and 135 nm, respectively. After 6 months of storage at ambient temperature, the particle size of the LYO and GIL samples also remained essentially unchanged, with a mean particle size of 142 nm and 146 nm, respectively. These unexpected results indicate a physically stable formulation after gamma irradiation of lyophilized nanoparticulate naproxen samples with respect to particle size.

Table 2 above, shows that the initial mean particle sizes of Formulation 2 was 175 nm. The mean particle size of the LYO and GIL samples increased to 239 nm and 224 nm, respectively. After 3 months of storage at ambient temperature, the LYO and GIL samples had a mean particle size of 247 nm and 202 nm, respectively. Surprisingly, after 6 months of storage at ambient temperature, the LYO and GIL samples had a mean particle size of 225 nm and 235 nm, respectively, indicating that no substantial agglomeration of the naproxen particles occurred after being exposed to gamma radiation.

E. Osmolality and pH Results

The osmolality values of Formulation 1, listed above in Table 1, also remained constant at approximately 80-90 mOsm/kg over 6 months at ambient temperature for the lyophilized and GIL samples. Although these values were very low, they were comparable to the theoretical value for osmolality of 88 mOsm/kg. The theoretical osmolality was calculated by including contributions from naproxen, PVP, and sodium hydroxide.

Due to its osmolality value, Formulation 1 was found to be very hypotonic. For bolus injection, the formulation could be reformulated to incorporate a tonicity adjuster, such as 0.9% NaCl, 5% mannitol, or 5% dextrose, etc.

The initial pH of Formulation 1 was 7.0 for the NCD and 6.8 for the LYO and GIL samples. At the 3 months and 6 months timepoints at ambient temperature, the pH was 6.9 for both the LYO and GIL samples. The slight changes in pH were not an indication of instability because they were well within the variability limits of the pH meter.

As illustrated in Table 2 above, Formulation 2 generated higher osmolality values than Formulation 1. This result was expected due to the presence of L-Histidine in Formulation 2. Values ranged from 346 mmol/kg to 367 mmol/kg over the 6 months time interval. The experimental osmolality values were comparable to the theoretical value of 296 mmol/kg. The theoretical value was calculated by including ionic contributions from naproxen, PVP, and L-Histidine.

The measured values indicated that Formulation 2 was slightly hypertonic. If necessary, the histidine could be reduced to approximately 3.5% to provide an isotonic solution. The initial pH of Formulation 2 was 6.8 for the naproxen NCD and remained unchanged post-lyophilization and post-gamma irradiation. The pH for the LYO and GIL samples at 3 months and 6 months was 6.9. Therefore, the pH was very stable over the course of the study.

F. Chemical Stability Results

Assay results of Formulations 1 and 2 of the initial naproxen NCD, LYO, and GIL samples are illustrated in Table 3 below. The assay results were established by measuring the percent label claim (% LC) of samples of Formulations 1 and 2 initially and after three (3) months at two different storage temperatures (25° C./60% RH and 40° C./75% RH) by high performance liquid chromatography (HPLC).

The HPLC assay procedure included preparing samples and standards at 1 mg/mL in 70:30 acetonitrile:H₂O. The mobile phase was 65% [0.05M KH₂PO₄ adjusted to pH 3 with H₃PO₄+1% of glacial acetic acid]: 35% acetonitrile. The chromatographic conditions were as follows: wavelength of detection: 270 nm; run time=20 min.; injection volume=10 μL; column temperature=25° C. The chromatographic system was a Waters 2690 Separations Module; Waters 2487 Dual Wavelength Detector; Waters Millennium 32 Chromatography Manager; and the column was a Brownlee RP8 Spheri-5 C8.

TABLE 3
Naproxen Percent Label Claim (% LC) Summary

| | Initial/ | | | |
|---|---|---|---|---|
| Type | 3 month | Temp. | % LC Average | % RSD (n = 4) |
| NCD¹ | Initial | 25° C./60% RH | 101.2 | 0.5 |
| LYO¹ | Initial | 25° C./60% RH | 100.4 | 0.9 |
| LYO¹ | 3 month | 25° C./60% RH | 100.9 | 0.3 |
| LYO¹ | 3 month | 40° C./75% RH | 100.7 | 1.1 |
| GIL¹ | Initial | 25° C./60% RH | 100.5 | 0.6 |
| GIL¹ | 3 month | 25° C./60% RH | 96.6 | 1.3 |
| GIL¹ | 3 month | 40% C/75% RH | 102.7 | 1.5 |
| NCD² | Initial | 25° C./60% RH | 73.5 | 0.4 |
| LYO² | Initial | 25° C./60% RH | 72.6 | 1.1 |
| LYO² | 3 month | 25° C./60% RH | 73.1 | 0.4 |
| LYO² | 3 month | 40° C./75% RH | 72.0 | 1.4 |
| GIL² | Initial | 25° C./60% RH | 90.5³ | 0.1 (n = 2) |
| GIL² | Initial | 25° C./60% RH | 72.3⁴ | 1.3 (n = 2) |
| GIL² | 3 month | 25° C./60% RH | 73.0 | 1.3 |
| GIL² | 3 month | 40° C./75% RH | 71.5 | 0.4 |

¹20% Naproxen + 2% PVP + NaOH (Formulation 1)
NCD - Pre-lyophilized NCD
LYO - Lyophilized NCD
GIL—Gamma Irradiated Lyophilized NCD
²20% Naproxen + 2% PVP + 4% L-Histidine (Formulation 2)
NCD - Pre-lyophilized NCD
LYO - Lyophilized NCD
GIL - Gamma Irradiated Lyophilized NCD
³Sample was not reconstituted prior to weighing.
⁴Sample was reconstituted prior to weighing.

As illustrated in Table 3 above, the initial NCD had a naproxen label claim of 101%. The naproxen LYO and GIL samples had initial label claims of 100%. After 3 months storage at 25° C./60% relative humidity (RH), the label claim of the LYO and GIL samples was 101% and 96.79%, respectively. After three months of 40° C./75% RH, the LYO and GIL samples had a label claim of 101% and 103%, respectively. Surprisingly, the assay results illustrated in Table 3 indicate that Formulation 1 was stable at all conditions of storage over 3 months post-gamma irradiation.

The assay results of Formulation 2 are also listed in Table 3 above. Formulation 2, containing L-Histidine, yielded an initial % label claim for the naproxen NCD of 73.5%. For the LYO and GIL samples, a label claim of 72.6% and 72.3%, respectively, was obtained.

The HPLC procedure was performed by reconstituting the samples with sterile water for injection (SWFI), followed by dilution in the mobile phase. Because the % label claim was low, an alternate sample preparation was also investigated. A GIL sample was analyzed by removing the dry powder material from the vial and diluting directly with the mobile phase. With this method, a label claim of 90.5% was obtained. Without being bound by theory it is believed that the low label claim values are due to compounding error. Assuming an initial concentration of naproxen of 14% and not 20%, with water making up the balance, a value of 91% label claim for the non-reconstituted sample was consistent with the measured value of 90.5%. This value was also consistent with a label claim of 70% for the reconstituted LYO samples.

The degradation products in Formulations 1 and 2 were quantified by HPLC and the resulting values were based on % peak area as illustrated in Tables 4, 5, and 6 below. The unknown degradation products were all below 0.1 % with the exception of Unknown 7, which appears only in the GIL samples at levels ranging from 0.23% to 0.34%. It appears that this degradation product was generated at low levels of gamma irradiation.

TABLE 4
Percent Degradation of Initial Naproxen Samples at 25° C.

| Type | Naproxen | Unknown³ 1 | Unknown 2 | Unknown 3 | Unknown 5 | Unknown 6 | Unknown 7 | Unknown 8 |
|---|---|---|---|---|---|---|---|---|
| NCD | 99.92 | 0.02 | 0.04 | 0.02 | | 0.02 | | |
| LYO | 99.92 | 0.02 | 0.04 | 0.02 | | | | |
| GIL | 99.49 | 0.02 | 0.06 | 0.03 | 0.06 | 0.04 | 0.27 | 0.09 |
| NCD | 99.91 | 0.02 | 0.04 | 0.02 | | 0.02 | | |
| LYO | 99.91 | 0.02 | 0.04 | 0.02 | | | | |
| GIL | 99.50 | 0.02 | 0.06 | 0.02 | 0.07 | 0.04 | 0.25 | |

¹Formulation 1: 20% Naproxen + 2% PVP + NaOH
²Formulation 2: 20% Naproxen + 2% PVP + 4% L-Histidine
³Unknown values are in units of % peak area.

TABLE 5
Percent Degradation of 3 Month Stability Samples at 25° C./60% RH

| Type | Naproxen | Unknown³ 1 | Unknown 2 | Unknown 3 | Unknown 5 | Unknown 6 | Unknown 7 | Unknown 8 |
|---|---|---|---|---|---|---|---|---|
| LYO¹ | 99.90 | 0.02 | 0.04 | 0.02 | | 0.06 | | |
| GIL¹ | 99.41 | 0.02 | 0.06 | 0.03 | 0.07 | 0.04 | 0.29 | 0.02 |
| LYO² | 99.90 | 0.02 | 0.04 | 0.02 | | | | |
| GIL² | 99.48 | 0.02 | 0.05 | 0.02 | 0.07 | 0.03 | 0.23 | 0.06 |

¹Formulation 1: 20% Naproxen + 2% PVP + NaOH
²Fomulation 2: 20% Naproxen + 2% PVP + 4% L-Histidine
³Unknown values are in units of % peak area.

TABLE 6
Percent Degradation of 3 Month Stability Samples 40° C./75% RH

| Type | Naproxen | Unknown³ 1 | Unknown 2 | Unknown 3 | Unknown 5 | Unknown 6 | Unknown 7 | Unknown 8 |
|---|---|---|---|---|---|---|---|---|
| LYO | 99.89 | 0.02 | 0.04 | 0.02 | 0.01 | 0.02 | | |
| GIL | 99.37 | 0.02 | 0.06 | 0.03 | 0.07 | 0.03 | 0.34 | 0.06 |
| LYO | 99.90 | 0.02 | 0.04 | 0.02 | | | 0.01 | |
| GIL | 99.42 | 0.02 | 0.05 | 0.02 | 0.09 | 0.04 | 0.27 | 0.03 |

¹Formulation 1-20% Naproxen + 2% PVP + NaOH
²Formulation 2-20% Naproxen + 2% PVP + 4% L-Histidine
³Unknown values are in units of % peak area.

Without being bound by theory, it is believed that the source of the degradation product is from the naproxen active pharmaceutical ingredient (API). Gamma irradiation dosing studies of naproxen API were conducted with doses of 0, 5, 10, 15, 25, 30, 40, and 50 kGy. A degradation product was detected in the API at the same Relative Retention Time (RRT) as the degradant in the GIL samples. The degradant in the API was present in all samples at all radiation doses tested. There was a direct relationship between the gamma dose and quantity of the degradation product. The results of this testing are shown below in Table 7.

TABLE 7
Naproxen Assay Results

| | | | | | | | |
|---|---|---|---|---|---|---|---|
| Control | 5 kGy | 10 kGy | 15 kGy | 25 kGy | 30 kGy | 40 kGy | 50 kGy |

Naproxen (API) (% w/w)

| | | | | | | | | |
|---|---|---|---|---|---|---|---|---|
| Average | 99.07 | 96.38 | 100.82 | 100.44 | 100.08 | 100.47 | 99.14 | 99.49 |
| Relative | 100.00 | 97.28 | 101.77 | 101.38 | 101.02 | 101.41 | 100.07 | 100.42 |
| to Control | | | | | | | | |

Unknown Degradants in Naproxen API (% by Area)

| | | | | | | | | |
|---|---|---|---|---|---|---|---|---|
| RRT | n/a | 0.07 | 0.07 | 0.09 | 0.12 | 0.14 | 0.16 | 0.18 |
| 1.07 | | | | | | | | |
| RRT | n/a | 0.10 | 0.10 | 0.14 | 0.21 | 0.23 | 0.27 | 0.33 |
| 1.35 | | | | | | | | |

Lyophile (% w/w)

| | | | | | | | | |
|---|---|---|---|---|---|---|---|---|
| Average | 90.00 | 90.37 | 89.98 | 89.71 | 89.52 | 90.57 | 90.19 | 89.53 |
| Relative | 100.00 | 100.41 | 99.98 | 99.68 | 99.47 | 100.63 | 100.21 | 99.48 |
| to Control | | | | | | | | |

Nanoparticulate Naproxen Dispersion (NCD) (% w/w)

| | | | | | | | | |
|---|---|---|---|---|---|---|---|---|
| Average | 39.90 | 39.66 | 40.16 | 39.97 | 39.80 | 39.83 | 39.66 | 39.71 |
| Relative | 100.00 | 99.40 | 100.65 | 100.18 | 99.75 | 99.82 | 99.40 | 99.52 |
| to Control | | | | | | | | |

G. Weight Distribution

The concentration and molecular weight (Mw) distributions of PVP in the naproxen NCD samples were determined by a Gel Permeation Chromatography (GPC) method. A Waters 2690 Separation Module, Viscotec 300TDA detector, and TSK-gel G3000PWXL column were used to perform the analysis. PEO 26K standard was used to calibrate the 300TDA detector. The intrinsic viscosity (dη/dc) of PVP in different mobile phases was measured based on the PVP starting material, and the dη/dc value was then used to calculate the molecular weight distributions and concentrations of PVP in the formulations by the TriSEC software. The concentration of PVP determined using an external standard is close to the concentration determined by means of the TriSEC software.

Two GPC methods were used to determine the corresponding initial average molecular weight of PVP in naproxen Formulations 1 and 2. The sample preparation and other conditions of the two GPC methods are set forth in Table 8 below.

TABLE 8
GPC methods used for PVP analysis of Naproxen samples

| GPC Method | Sample Preparation |
|---|---|
| Method 1 | Mobile phase: 100 ppm NaN₃/H₂O, 0.8/min, temp. 30° C. |
| | Sample preparation: Naproxen NCD was diluted with |
| | 100 ppm NaN₃ to make about 4 mg/mL PVP, |
| | then the sample was ultracentrifuged at 80 K for 15 min. |
| | (10° C.). 100 μL of the supernatant |
| | was injected into the column. |
| | Total run time: 50 min. |
| Method 2 | Mobile phase: 30% (w/w) MeOH/H₂O, 0.5 mL/min, |
| | temp. 30° C. Sample preparation: Naproxen NCD |
| | was dissolved in methanol, then H₂O was added to |
| | make a 30% solution, which was then filtered |
| | through 0.45 μL of the filtrate into the column. |
| | Total run time: 90 min. |

A comparison of the results obtained by the two GPC methods is shown in Tables 9a and 9b below. The average initial percent label claim (% LC) of PVP is listed in Table 9a and the corresponding initial average molecular weight of PVP is listed in Table 9b.

Comparison of GPC Method 1 and Method 2 on Naproxen Samples

TABLE 9a
Average % LC of PVP Using Methods
1 & 2 to Analyze Initial Naproxen Samples

| | | |
|---|---|---|
| Formulation | Method 1 | Method 2³ |

| Type | Initial | % RSD(n = 4) | Initial | % RSD |
|---|---|---|---|---|
| NCD¹ | 77 | 1.4 | 93 | 0.3⁴ |
| LYO¹ | 78 | 0.3 | 88 | 6.8 |
| GIL¹ | 74 | 5.5 | 92 | 0.5 |
| NCD² | 99 | 1.5 | 100 | 0.0⁵ |
| LYO² | 101 | 0.5 | 103 | 2.5 |
| GIL² | 100 | 0.4 | 98 | 2.0 |

¹Formulation 1: 20% naproxen + 2% PVP + NaOH
²Formulation 2: 20% naproxen + 2% PVP + 4% L-Histidine
³Combination of data collected on Jan. 3, 2002 and Feb. 01, 2002, n = 6
⁴Data collected on Feb. 01, 2002 only, n = 3
⁵Data collected on Feb. 01, 2002 only, n = 2

TABLE 9b
Average Mw of PVP Using Methods 1 & 2 to Analyze Naproxen Samples

| | Method 1 | | | | | |
|---|---|---|---|---|---|---|
| Formulation | | | % RSD | Method 2³ | | |
| Type | Mw | % Mw | (n = −4) | Mw | % Mw⁶ | % RSD |
| NCD¹ | 2.0E+03 | 72 | 2.7 | 2.9E+03 | 87 | 1.8⁴ |
| LYO¹ | 1.9E+03 | 67 | 5.6 | 2.8E+03 | 85 | 11.2 |
| GIL¹ | 1.9E+03 | 68 | 1.8 | 2.7E+03 | 81 | 2.3 |
| NCD² | 2.4E+03 | 85 | 5.4 | 3.0E+03 | 89 | 3.6⁵ |
| LYO² | 2.4E+03 | 85 | 0.3 | 2.9E+03 | 86 | 7.5 |
| GIL² | 2.4E+03 | 84 | 2.7 | 2.7E+03 | 80 | 5.3 |
| PVP Formulation 2 | 2.9E+03 | | 4.3(n = 21) | 3.3E+03 | | 4.0 |

¹Formulation 1: 20% naproxen + 2% PVP + NaOH
²Formulation 2: 20% naproxen + 2% PVP + 4% L-Histidine
³Combination of data collected on Jan. 3, 2002 and Feb. 01, 2002, n = 6
⁴Data collected on Feb. 01, 2002 only, n = 3
⁵Data collected on Feb. 01, 2002 only, n = 2
⁶% Mw = Relative Average Molecular Weight compared to that of PVP in starting material.

The initial and 3 month stability data as determined by Method 1 for % LC of PVP and average PVP molecular weight in the naproxen NCD formulation samples are shown in Tables 10a and 10b below. Method 1, which was used to report stability data, was the preferred method since the run times were much shorter than the values obtained with Method 2, and Method 1 was thus more robust than Method 2.

It appears that the samples containing naproxen NCD had a higher affinity for the GPC column in Method 2, and did not elute for over 2 hours with variability in run times. The method development was necessary to determine if a column designed for an organic mobile phase would be more appropriate for Method 2 to reduce the run time.

Without being bound by theory, it is assumed that in Method 1 the dilution in water followed by centrifugation only quantitated the free polymer, i.e., the material that was not associated with the naproxen particles. The technique in Method 2 improves recovery of PVP due to minimal sample manipulation and, thus, is more representative of the PVP total distribution present in the sample.

This theory can explain the increase in the molecular weight distribution seen in Method 2 as compared to Method 1, and would indicate that the higher molecular weight chains are preferentially associating with the naproxen particles. In Method 2 more PVP was liberated from the naproxen particle surfaces into solution, therefore increasing the amount of higher molecular weight species present in solution to yield the higher molecular weight distributions.

H. Comparison of GPC Methods 1 and 2

Quantification of PVP present in each sample and the molecular weight of PVP was determined via GPC analysis. Two methods were used to analyze the Mw of PVP. Method 1, using water and ultracentrifugation, provided reproducible results, but could not be used to recover all of the PVP in the naproxen samples. Method 2, using methanol (MeOH), appeared to liberate all PVP and represented the total distribution of PVP present in the sample, but was not as reproducible.

PVP results obtained according to Method 1 are set forth in Table 9a for the naproxen NCD, LYO, and GIL samples of Formulation 2, which had approximately 100% label claim (LC). For the naproxen NCD, LYO, and GIL samples of Formulation 1, the PVP % label claim ranged from 74% to 78%. Without being bound by theory, the fact that the naproxen NCD, LYO, and GIL samples of Formulation 1 had lower PVP recoveries was due to the PVP being more tightly associated with the naproxen crystals in Formulation 1, whereas in the naproxen NCD, LYO, and GIL samples of Formulation 2, L-Histidine was competing with the PVP for associating with the naproxen crystal surface, and thus more PVP could be recovered.

The % LC for the naproxen NCD, LYO, and GIL samples of Formulation 1 as determined by Method 2, were higher than values obtained by Method 1 (see Table 9a). However, the % LC results for the naproxen NCD, LYO, and GIL samples of Formulation 2 determined by Method 2 were very similar to % LC values obtained by following Method 1. This indicates that Method 2 was effecting the release of bound PVP from the naproxen samples of Formulation 1, and had no influence on the looser bound PVP found in the naproxen NCD, LYO, and GIL samples of Formulation 2 which contained L-Histidine.

Table 9b sets forth the average molecular weight (Mw) of PVP in the initial naproxen samples as determined by Methods 1 and 2. The % Mw was relative to the average molecular weight of the PVP of the starting material. The data of Table 9b indicates that Method 1 yields lower % Mw values, ranging from 67% to 72% for the naproxen NCD, LYO, and GIL samples of Formulation 1. For the naproxen NCD, LYO, and GIL samples of Formulation 2 at approximately 85%, the % Mw values were higher. As stated above, when using Method 1 not all the PVP was recovered.

The results obtained for % MW by following Method 2 are set forth in Table 9. The results are similar for the naproxen NCD samples of Formulations 1 and 2, which contained 87% and 89% Mw, respectively; the naproxen LYO samples of Formulations 1 and 2, which contain 85% and 86% Mw, respectively; and the naproxen GIL samples of Formulations 1 and 2, which contained 81% and 80% Mw, respectively.

Methods 1 and 2 provided comparable % LC and average Mw results for the naproxen NCD, LYO, and GIL samples of Formulation 2. Without being bound by theory, it is believed that this was due to weakened association of PVP with the naproxen particles when L-Histidine was present. Methods 1 and 2 generated different results for the naproxen NCD, LYO, and GIL samples of Formulation 1 due to stronger association of PVP and naproxen particles in the absence of L-Histidine.

Samples containing naproxen NCD had a higher affinity for the GPC column in Method 2, because the naproxen API peak did not elute for over 2 hours, with varying elution times. For this reason, Method 1 was used to analyze PVP stability data at initial and 3 month time intervals. This method provided the means to qualitatively compare PVP stability within a formulation over time.

I. PVP % LC and % Molecular Weight Stability Results

The PVP % LC results from Method 1 are presented in Table 10a below.

TABLE 10a
Average % LC of PVP Using Method 1
to Analyze NCD Stability Samples

| | Initial | | 3 months | |
|---|---|---|---|---|
| Type | % LC | % RSD (n = 4) | % LC | % RSD (n = 4) |
| NCD¹ | 77 | 1.4 | | |
| LYO¹ 25° C./60°% RH | 78 | 0.3 | 77 | 0.8 |
| LYO¹ 40° C./75°% RH | | | 77 | 1.3 |
| GIL¹ 25° C./60°% RH | 74 | 5.5 | 72 | 1.5 |
| GIL¹ 40° C./75°% RH | | | 76 | 6.8* |
| NCD² | 99 | 1.5 | | |
| LYO² 25° C./60°% RH | 101 | 0.5 | 97 | 2.8 |
| LYO² 40° C./75°% RH | | | 98 | 0.4 |
| GIL² 25° C./60°% RH | 100 | 0.4 | 97 | 1.4 |
| GIL² 40° C./75°% RH | | | 96 | 0.4 |

*n = 3
¹Formulation 1: 20% naproxen + 2% PVP + NaOH
²Formulation 2: 20% naproxen + 2% PVP + 4% L-Histidine

The % LC for the naproxen LYO and GIL samples of Formulation 1 remained unchanged over 3 months when stored at either 25° C./60% RH or 40° C./75% RH. All values were approximately 75% LC. Therefore, Formulation 1 was stable with respect to PVP content for 3 months and at all temperatures in the LYO and GIL samples.

The PVP % LC for the naproxen LYO and GIL samples of Formulation 2 also indicated a stable formulation as shown in Table 10a above. The results from the initial analysis and 3 month analysis were comparable at 25° C./60% RH and 40° C./75% RH, with all values being approximately 100% LC. The PVP content of the LYO and GIL samples of the L-Histidine containing Formulation 2 was constant over 3 months at all temperatures.

The % Mw of PVP for the naproxen NCD, LYO, and GIL samples of Formulation 1 is listed in Table 10b below. Again, the values were consistent over the 3 months and at the two temperatures, (25° C./60% RH and 40° C./75% RH), with values of approximately 70%. Formulation 1 was therefore stable with respect to PVP content.

TABLE 10b
Average Mw of PVP Using Method 1 to Analyze Naproxen Stability Samples

| Naproxen | Initial | | | 3 months | | |
|---|---|---|---|---|---|---|
| Formulations | | | % RSD | | | % RSD |
| Type | Mw³ | % Mw | (n = 4) | Mw | % Mw | (n = 4) |
| NCD¹ | 2.0E+03 | 72 | 2.7 | | | |
| LYO¹ 25° C./60°% RH | 1.9E+03 | 67 | 5.6 | 2.0E+03 | 69 | 1.9 |
| LYO¹ 40° C./75°% RH | | | | 2.0E+03 | 72 | 4.8 |
| GIL¹ 25° C./60°% RH | 1.9E+03 | 68 | 1.8 | 2.0E+03 | 70 | 2.2 |
| GIL¹ 40° C./75% RH | | | | 2.0E+03 | 71 | 1.7* |
| NCD² | 2.4E+03 | 85 | 5.4 | | | |
| LYO² 25° C./60°% RH | 2.4E+03 | 85 | 0.4 | 2.5E+03 | 85 | 2.0 |
| LYO² 40° C./75°% RH | | | | 2.6E+03 | 88 | 2.0 |
| GIL² 25° C./60°% RH | 2.4E+03 | 84 | 2.7 | 2.5E+03 | 86 | 0.5 |
| GIL² 40° C./75°% RH | | | | 2.5E+03 | 86 | 0.9 |
| PVP | 2.9E+03 | | 4.3** | 2.9E+03 | | 1.7* |

*n = 3,
**n = 21
¹Formulation 1: 20% naproxen + 2% PVP + NaOH
²Formulation 2: 20% naproxen + 2% PVP + 4% L-Histidine
³% Mw: Relative Average Molecular Weight compared to that of PVP starting material.

The naproxen NCD, LYO, and GIL samples of Formulation 2 also had constant % Mw values of approximately 86% for 3 months at 25° C./60% RH and 40° C./75% RH. This was also an indication of the acceptable stability of Formulation 2 (containing L-histidine) for 3 months at the indicated temperatures.

The above results illustrate that the naproxen NCD, LYO, and GIL samples of both Formulations 1 and 2 maintained the % LC and % MW of PVP when stored at 25° C./60% RH and 40° C./75% RH for 3 months.

J. Microbiological Testing

Microbiological testing, including Microbiological Limit Tests (MLT), Bacterial Endotoxins Test (BET), and sterility testing, was conducted on samples of Formulations 1 and 2.

BET results, conducted by LAL gel clot method (described in the United States Pharmacopeia (USP)), for samples of Formulations 1 and 2 are set forth in Table 11 below.

TABLE 11
Bacterial Endotoxin Testing Results
by LAL Gel Clot Method

| NCD/LYO/GIL | Dilution | Specification | Result |
|---|---|---|---|
| NCD¹ (5° C.) | 1:600 | Report results | <0.09 EU/mg |
| LYO¹ | 1:600 | Report results | <0.09 EU/mg |
| GIL¹ | 1:600 | Report results | <0.09 EU/mg |
| NCD² (5° C.) | 1:600 | Report results | <0.09 EU/mg |
| LYO² | 1:600 | Report results | <0.09 EU/mg |
| GIL² | 1:600 | Report results | <0.09 EU/mg |

¹Formulation 1: 20 % Naproxen + 2% PVP and NaOH
²Formulation 2: 20 % Naproxen + 2% PVP + 4% L-Histidine

The BET results obtained for all NCDs, LYO samples, and GIL samples were <0.09 EU/mg, which indicated that the samples contained acceptable, low levels of endotoxins.

MLT results were obtained from samples of both Formulations 1 and 2 using the NCDs and LYO (non-sterilized and lyophilized) samples of these formulations. The results for Formulations 1 and 2 are set forth in Table 12 below for the naproxen NCDs.

TABLE 12
Microbial Limits Testing*,** Results for 20% Naproxen
NCD formulations at 5° C.

| Organism | Specification | Result |
|---|---|---|
| Total Aerobic Count¹ | Report Results | <100 CFU/mL |
| Total Yeast & Mold Count¹ | Report Results | <100 CFU/mL |
| Escherichia coli¹ | Report Results | Absent |
| Salmonella species¹ | Report Results | Absent |
| Pseudomonas aeruginosa¹ | Report Results | Absent |
| Staphylococcus aureus¹ | Report Results | Absent |
| Total Aerobic Count² | Report Results | <100 CFU/mL |
| Total Yeast & Mold Count² | Report Results | 50 CFU/mL |
| Escherichia coli² | Report Results | Absent |
| Salmonella species² | Report Results | Absent |
| Pseudomonas aeruginosa² | Report Results | Absent |
| Staphylococcus aureus² | Report Results | Absent |

*Microbial Limits Testing - USP 24, Supplement 4 <61>.
**The dilution of Naproxen NCD Formulation to Growth Media is 1:100.
¹Formulation 1 = 20% Naproxen + 2% PVP and NaOH
²Formulation 2 = 20% Naproxen + 2% PVP + 4% L-Histidine

Table 13 below sets forth MLT results for lyophilized samples of Formulations 1 and 2.

TABLE 13
Microbal Limits Testing*,** Results for 20%
Naproxen LYO formulations

| Formulations | Organism | Specification | Result |
|---|---|---|---|
| Formulation 1 | Total Aerobic Count | Report Results | <100 CFU/mL |
| LYO | Total Yeast & | Report Results | <100 CFU/mL |
| | Mold Count | | |
| | Escherichia coli | Report Results | Absent |
| | Salmonella species | Report Results | Absent |
| | Pseudomonas | Report Results | Absent |
| | aeruginosa | | |
| | Staphylococcus aureus | Report Results | Absent |
| Formulation 2 | Total Aerobic Count | Report Results | <100 CFU/mL |
| LYO | Total Yeast & | Report Results | <100 CFU/mL |
| | Mold Count | | |
| | Escherichia coli | Report Results | Absent |
| | Salmonella species | Report Results | Absent |
| | Pseudomonas | Report Results | Absent |
| | aeruginosa | | |
| | Staphylococcus aureus | Report Results | Absent |

*Microbial Limits Testing - USP 24, Supplement 4 <61>.
**The dilution of Naproxen NCD Formulation to Growth Media is 1:100.

The results of Tables 12 and 13 above indicate the absence of four indicator organisms, and <100 CFU/mL for both yeasts and mold and aerobic counts.

Sterility analyses were conducted on the naproxen LYO and GIL samples of Formulations 1 and 2. The results are set forth in Tables 14 and 15 below.

TABLE 14
Bacteriostasis/Fungistasis Testing¹
Results for 20% Naproxen GIL formulations

| | | Reference | | |
|---|---|---|---|---|
| | | Plate | | |
| | | CFU's | | |
| | | (Plate 1/ | Specifi- | |
| Formulations | Organism | Plate 2) | cation² | Result |
| Formulation 1 | Staphylococcus aureus | 36/63 | Growth | Growth |
| GIL | Pseudomonas aeruginosa | 24/25 | Growth | Growth |
| | Bacillus subtillis | 44/35 | Growth | Growth |
| | Clostridium sporogenes | 7/6 | Growth | Growth |
| | Aspergillius niger | 64/57 | Growth | Growth |
| | Candida albicans | 56/46 | Growth | Growth |
| Formulation 2 | Staphylococcus aureus | 36/63 | Growth | Growth |
| GIL | Pseudomonas aeruginosa | 24/25 | Growth | Growth |
| | Bacillus subtillis | 44/35 | Growth | Growth |
| | Clostridium sporogenes | 7/6 | Growth | Growth |
| | Aspergillius niger | 64/57 | Growth | Growth |
| | Candida albicans | 56/46 | Growth | Growth |

¹Bacteriostasis/Fungistasis Testing - USP 24, Supplement 4 <71>.
²Growth medium was fluid thioglycollate medium (FTM) and tryptic soy broth (TSB) both with and without product (naproxen GIL)

TABLE 15
Sterility Testing¹ Results for 20% Naproxen GIL formulations.

| Formulations | Media | Specification | Result |
|---|---|---|---|
| Formulation 1 | TSB with product | Report Results | No Growth |
| GIL | FTM with product | Report Results | No Growth |
| Formulation 2 | TSB² with product | Report Results | No Growth |
| GIL | FTM³ with product | Report Results | No Growth |

¹Sterility Testing - USP 24, Supplement 4 <71> Sterility Tests.
²TSB—Tryptic soy broth
³FTM—Fluid thioglycollate medium

The results set forth in Table 14 above show that bacteria and fungi will grow in the presence of naproxen. The growth/recovery experiment is a validation of the MLT. Thus, simply stated, the MLT, BET, and sterility tests were validated in accordance with requirements of USP 24. Table 15 above shows that no bacterial growth secured for the gamma irradiated samples of Formulations 1 and 2. Therefore, the process of terminal sterilization by gamma irradiation provided by the present invention produced a sterile product. Both Formulations 1 and 2 met the sterility requirements set forth by USP 24<71>, Supplement 4.

The results obtained in Examples 1 and 2 above, illustrate that sterile nanoparticulate naproxen formulations can be obtained by applying the method of the invention. Results obtained for particle size, osmolality, pH, percent label claim, molecular weight of PVP, as well as the microbiological and sterility results, indicate that by using the method of the present invention terminally sterilized nanoparticulate naproxen formulations are obtained. Moreover, such formulations are suitable for parenteral administration.

### EXAMPLE 3

The purpose of this example was to prepare a more concentrated nanoparticulate active agent formulation and to evaluate the overall stability pre-lyophilization, post-lyophilization, and post-gamma radiation of the concentrated formulation.

A nanoparticulate naproxen dispersion was prepared comprised of 40% naproxen, 4% PVP, with a pH adjusted to 7.0 using a 1 N solution of NaOH.

60 g of PVP (Kollidon® 12 PF, BASF) was stirred into approximately 700 g of sterile water for injection (Abbott Laboratories) in a 2 L stainless steel recirculation vessel until the PVP dissolved. 600 g of naproxen (Alfa Chemical) was stirred into the resulting solution until the drug was thoroughly wetted. The pH of the resulting slurry was adjusted to approximately 7 by adding 82 g of 1N NaOH solution. The remaining 58 g of sterile water for injection was added to the slurry while stirring with an overhead mixer.

Subsequently, PolyMill™-200 (DOW) milling media was charged via a vacuum into a 600 cc continuous feed milling chamber of a DYNO®-Mill (Type: KDL, manufactured by Willy Bachofen, AG Maschinenfabrik). The naproxen containing slurry was milled for approximately 2 hours. A filter apparatus and a 100 μm mesh screen were used to harvest the nanoparticulate naproxen dispersion. The bulk dispersion was filtered through a 5 μm PolyCap™ 36 HD (Whatman) filter into a receiver vessel. 1239 g of nanoparticulate naproxen dispersion was collected having a mean particle size of 89 nm.

Approximately 1.5 g of the above formulation was dispensed into 3 mL autoclaved glass vials (West Co.) which were stoppered with autoclaved 13 mm 4432/50 (B2-44 coating) gray butyl stoppers (West Co.).

The stoppered samples had their stoppers loosened so that air could enter and were then transferred to a Dura-Stop™ Lyophilizer (FTS™ Systems), where the samples were lyophilized. Prior to removing the samples from the lyophilizer, they were stoppered under vacuum. The samples were then removed from the lyophilizer and crimped for the sterilization step.

Thereafter, the lyophilized samples were terminally sterilized via gamma irradiation at a dose of 25 kGray.

The formulation prepared above was tested for particle size, optical microscopy, pH, osmolality, and assay to ascertain that the formulation degradation profile is acceptable. Upon observation by optical microscopy, the formulation was homogeneous and free-flowing.

A. Particle Size Analysis

Naproxen pre-lyophilization, post-lyophilization, and post-gamma irradiation samples were analyzed for particle size using a Horiba LA-910 Static Light Scattering Particle Analyzer. Each sample was measured without sonication and following one minute of sonication to determine whether or not the dispersions were aggregated. The reported values from the histograms include Dₘₑₐₙ and D₉₀ particle size. The particle size results for all samples are set forth in Table 16.

B. pH Measurement

The pH values of all naproxen samples (pre-lyophilization, post-lyophilization, and post-gamma irradiation) were measured using a Beckman Φ 720 pH meter. The pH meter was calibrated prior to use with pH=4 and pH=7 calibration buffer solutions. The pH results for all samples are set forth in Table 16.

C. Osmolality Measurements

The osmolality values of the naproxen post-lyophilization and post-gamma irradiation samples were measured using a Wescor Vapro® Vapor Pressure Osmometer. A 10 μL sample of reconstituted nanoparticulate naproxen dispersion was used for each measurement. The osmometer was calibrated prior to use with a 290 mmol/kg standard solution. The osmolality results obtained for all samples are set forth in Table 16 below.

TABLE 16
Particle Size, pH and Osmolality Stability of 40% Naproxen + 4% PVP + NaOH

| | | Particle Size | | Particle Size | | | | |
|---|---|---|---|---|---|---|---|---|
| | | D Mean (nm) | | D 90 (nm) | | | | |
| | | | 1 min. | | 1 min. | | Osmolality | |
| Type | Time/Temp. | no son. | son. | no son. | son. | pH | Mosm/kg | Observations |
| NCD | Initial | 89 | 89 | 124 | 126 | 6.8 | 166 | No aggregation or large |
| | | | | | | | | particles |
| | 24 hr | 94 | 95 | 133 | 134 | — | — | No aggregation or large |
| | 5° C. | | | | | | | particles |
| LYO | Initial | 128 | 129 | 169 | 169 | 6.8 | 139 | No aggregation or large |
| | | | | | | | | particles |
| | 1 mo. | 102 | 102 | 146 | 146 | 6.9 | 160 | No aggregation or large |
| | 25° C./60% RH | | | | | | | particles |
| | 1 mo. | 14090 | 4633 | 40785 | 15548 | — | — | Aggregation |
| | 40° C./75% RH | | | | | | | |
| | 7 mo. | 103 | 125 | 149 | 166 | — | — | No aggregation or large |
| | 25 ° C./60% RH | | | | | | | particles |
| | 7 mo. | 39017 | 13811 | 112645 | 35967 | — | — | Aggregation |
| | 40° C./75% RH | | | | | | | |
| GIL | Initial | 126 | 126 | 166 | 166 | 6.8 | 144 | No aggregation or large |
| | | | | | | | | particles |
| | 1 mo. | 105 | 104 | 149 | 147 | 6.9 | 180 | No aggregation or large |
| | 25° C./60% RH | | | | | | | particles |
| | 1 mo. | 17284 | 8749 | 47617 | 21754 | — | — | Aggregation |
| | 40° C./75% RH | | | | | | | |
| | 7 mo. | 18357 | 10631 | 61038 | 30139 | — | — | Aggregation |
| | 25° C./60% RH | | | | | | | |
| | 7 mo. | 28617 | 11701 | 80053 | 27729 | — | — | Aggregation |
| | 40° C./75% RH | | | | | | | |

*no son. - without sonication

D. Particle Size and Optical Results

From Table 16 above, it is surprisingly apparent that the initial mean particle sizes of the naproxen lyophilized sample remained unchanged, with values of 128 nm & 126 nm for the LYO and GIL samples, respectively. This was relatively comparable to the initial naproxen NCD particle size of 89 nm.

After one-month storage at ambient temperature, the naproxen particle sizes of both the LYO and GIL samples essentially remained unchanged at 102 nm & 105 nm, respectively. However, at an elevated temperature of 40° C./75% RH at one month, both of the LYO and GIL samples exhibited an increase in particle size due to severe aggregation. The mean particle size for the LYO and GIL samples was 14,090 nm and 17,284 nm, respectively.

At seven months storage the particle size becomes unstable even at ambient temperature. The unsonicated mean particle size for the LYO and GIL samples was 103 nm and 18,357 nm, respectively. The LYO sample showed slight instability by increasing in size to 125 nm with sonication. Both the LYO and GIL samples at 40° C./75% RH were aggregated at seven months.

E. Osmolality and pH Results

Osmolality values for the formulation are listed in Table 16. The osmolality values remained relatively constant at approximately 139 mOsm/kg to 180 mOsm/kg when stored for one month at ambient temperature. Although all of these values are low, they are higher than the theoretical osmolality value of 43.64 mOsm/kg. The theoretical osmolality was calculated by including contributions from naproxen and PVP.

This formulation is hypotonic. If the formulation is intended for bolus injection, it may need to be reformulated to incorporate a tonicity adjuster, such as 0.9% NaCl, 5% mannitol, 5% dextrose, etc.

The initial pH for all naproxen samples (NCD, LYO, and GIL) was 6.8. For the LYO and GIL samples the pH was 6.9 at one month at ambient temperature (see Table 16). The slight changes are not an indication of instability because they are well within the variability limits of the pH meter.

F. Chemical Stability Results

Assay results for the naproxen NCD, LYO, and GIL samples at the initial timepoint are illustrated in Table 17 below. The assay results were established by measuring the percent label claim (% LC) of samples of the formulation at ambient temperature by high performance liquid chromatography (HPLC).

The HPLC assay procedure included preparing samples and standards at 1 mg/mL in 70:30 acetonitrile:H₂O. The mobile phase was 65% [0.05M KH₂PO₄ adjusted to pH 3 with H₃PO₄+1% of glacial acetic acid]: 35% acetonitrile. The chromatographic conditions were as follows: wavelength of detection: 270 nm; run time=20 min.; injection volume=10 μL; and column temperature=25° C. The chromatographic system was a Waters 2690 Separations Module; Waters 2487 Dual Wavelength Detector; Waters Millennium 32 Chromatography Manager; and the column was a Brownlee RP8 Spheri-5 C8.

TABLE 17
Initial Naproxen Percent Label Claim (% LC) at 25° C.

| | % LC | % RSD |
|---|---|---|
| TYPE | Average | (n = 4) |
| NCD | 100.0 | 4.7 |
| LYO | 101.3 | 1.0 |
| GIL | 101.4 | 0.9 |

Assay results at the initial timepoint are shown in Table 17. The initial NCD had a label claim average of 100%. The LYO and GIL samples had initial label claims of 101.3% and 101.4%, respectively. The data illustrates that the formulation is stable at initial ambient temperature, both pre- and post-gamma irradiation.

The degradation products in the 40% naproxen+4% PVP+NaOH formulation were quantified by HPLC and values based on % peak area. Results are presented in Table 18 below. The unknown degradation products are all below 0.1%, with the exception of Unknown 7, which appears only in the GIL sample at a level of 0.21%. It appears this degradation product is generated upon gamma irradiation, but at low levels.

TABLE 18
Percent Degradation of Naproxen Initial Samples at 25° C.

| | | Unknown | Unknown | Unknown | Unknown | Unknown | Unknown | Unknown | Unknown | Unknown |
|---|---|---|---|---|---|---|---|---|---|---|
| Type | Naproxen | 1 | 2 | 3 | 4 | 5 | 6 | 7 | 8 | 9 |
| NCD | 99.97 | 0.02 | 0.06 | 0.03 | | | | | | |
| LYO | 101.30 | 0.02 | 0.06 | 0.03 | | | | | | |
| GIL | 101.35 | 0.02 | 0.07 | 0.04 | | 0.06 | 0.02 | 0.21 | 0.06 | |

Without being bound by theory, it is believed that the source of the degradation product is from the active naproxen pharmaceutical ingredient (API). Gamma irradiation dosing studies of naproxen API were conducted with doses of 0, 5, 10, 15, 25, 30, 40, and 50 kGy. A degradation product was detected in the naproxen API at the same Relative Retention Time (RRT) as the degradant in the GIL samples. The degradant in the naproxen API was present in all samples at all radiation doses tested. There was a direct relationship between the gamma dose and quantity of the degradation product. Refer to Example 2, Table 7 for the results of this testing.

### EXAMPLE 4

The purpose of this example was to prepare a nanoparticulate naproxen formulation containing PVP K17 instead of PVP K12. The overall stability pre-lyophilization, post-lyophilization, and post-gamma radiation was evaluated. A nanoparticulate naproxen dispersion was prepared comprised of 40% naproxen, 4% PVP, and 5% sucrose, with a pH adjusted to 7.0 using a IN solution of NaOH.

54.5 g of PVP (Kollidon® 17 PF, BASF) was stirred into approximately 750 g of sterile water for injection (Abbott Laboratories) in a 2 L stainless steel recirculation vessel until the PVP stabilizer dissolved. 545.5 g of naproxen (Alfa Chemical) was stirred into the resulting solution until the drug was thoroughly wetted. The pH of the resulting slurry was adjusted to approximately 7 by adding 2 g of NaOH. The remaining 25 g of sterile water for injection was added to the slurry while stirring with an overhead mixer.

Subsequently, PolyMill™-200 (DOW) milling media was charged via a vacuum into a 600 cc continuous feed milling chamber of a DYNO®-Mill (Type: KDL, manufactured by Willy Bachofen, AG Maschinenfabrik). The naproxen containing slurry was milled for approximately 2 hours. The mill was stopped and 68.2 g of sucrose (Mallinckrodt) was added to the slurry and mixed for approximately 5 to 10 minutes with an overhead mixer. The slurry was milled for an additional 10 to 15 minutes. An autoclaved filter apparatus and a 100 μm mesh screen were used to harvest the nanoparticulate naproxen dispersion. The bulk dispersion was filtered through a 10 μm PolyCap™ 75 HD (Whatman) filter into an autoclaved receiver vessel. This material was subsequently filtered with a 1 μm PolyCap™ 75 HD (Whatman) filter into an autoclaved receiver vessel. 894 g of nanoparticulate naproxen dispersion was collected having a mean particle size of 139 nm.

Approximately 2.75 g of the above formulation was dispensed into 5 mL autoclaved glass vials (West Co.) which were stoppered with autoclaved 20 mm 4432/50 (B2-44 coating) gray butyl stoppers (West Co.).

The stoppered samples had their stoppers loosened so that air could enter and were then transferred to a Dura-Stop™ Lyophilizer (FTS™ Systems) where the samples were lyophilized. Prior to removing the samples from the lyophilizer, they were stoppered under vacuum. The samples were then removed from the lyophilizer and crimped for the sterilization step.

Thereafter, the lyophilized samples were terminally sterilized via gamma irradiation at a dose of 25 kGray.

Results:

The naproxen formulation prepared above was tested for particle size, optical microscopy, pH, osmolality, and assay to ascertain that the formulation degradation profile is acceptable. The samples were stored for 6 months at 25° C. and 40° C./75% RH, and samples were tested at Initial, 2, 4, 8, and 12 weeks, and 6 month timepoints.

A. Particle Size Analysis

Naproxen pre-lyophilization, post-lyophilization and post-gamma irradiation samples were analyzed for particle size using a Horiba LA-910 Static Light Scattering Particle Analyzer. Each sample was measured without sonication and following one minute of sonication to determine whether or not the dispersions were aggregated. The reported values from the histograms include Dₘₑₐₙ and D₉₀ particle size. The naproxen particle size results for all samples are set forth in Table 19.

B. pH Measurement

The pH values of all naproxen samples (pre-lyophilization, post-lyophilization, and post-gamma irradiation) were measured using a Beckman Φ 720 pH meter. The pH meter was calibrated prior to use with pH=4 and pH=7 calibration buffer solutions. The pH results for all samples are set forth in Table 19.

C. Osmolality Measurements

The osmolality values of the naproxen post-lyophilization and post-gamma irradiation samples were measured using a Wescor Vapro® Vapor Pressure Osmometer. A 10 μL sample of reconstituted nanoparticulate naproxen dispersion was used for each measurement. The osmometer was calibrated prior to use with a 290 mmol/kg standard solution. The osmolality results obtained for all samples are set forth in Table 19 below.

TABLE 19
Particle Size, pH and Osmolality Stability of
40% Naproxen + 4% PVP + 5% Sucrose + NaOH

| | | Mean (nm) | | D90 (nm) | | | | |
|---|---|---|---|---|---|---|---|---|
| | | no | 1 min. | no | 1 min. | | Osmolality | |
| Type | Time | son. | son. | son. | son. | pH | mOs mol/kg | Observations |
| NCD | Post 1 μm | 139 | 140 | 182 | 184 | 6.6 | — | Small particles, no |
| | filtration | | | | | | | aggregation or |
| | | | | | | | | crystal growth |
| | 24 hr @ 5° C. | 139 | 140 | 183 | 185 | — | — | — |
| LYO | Initial | 142 | 142 | 189 | 188 | 6.6 | 317 | Small particles, no |
| | | | | | | | | aggregation or |
| | | | | | | | | crystal growth |
| LYO | 2 week | 146 | 147 | 194 | 194 | 6.6 | 329 | Small particles, no |
| | (25° C.) | | | | | | | aggregation or |
| | | | | | | | | crystal growth |
| LYO | 2 week | 145 | 150 | 192 | 199 | 6.6 | 334 | Small particles, no |
| | (40° C.) | | | | | | | aggregation or |
| | | | | | | | | crystal growth |
| LYO | 4 Week | 147 | 148 | 194 | 195 | 6.6 | 333 | Crystal growth |
| | (25° C.) | | | | | | | ˜5 μm |
| LYO | 4 Week | 150 | 151 | 201 | 201 | 6.6 | 312 | Crystal growth |
| | (40° C.) | | | | | | | ˜5-8 μm |
| LYO | 8 Week | 139 | 139 | 185 | 184 | 6.7 | 316 | Crystal growth |
| | (25° C.) | | | | | | | ˜5 μm |
| LYO | 8 Week | 142 | 142 | 188 | 188 | 6.7 | 315 | Crystal growth |
| | (40° C.) | | | | | | | ˜5 μm |
| LYO | 12 Week | 141 | 141 | 187 | 187 | 6.6 | 623 | Slight crystal |
| Recon w/ | (25° C.) | | | | | | | growth |
| NaCl** | | | | | | | | Few particles |
| | | | | | | | | >5 um |
| LYO | 12 Week | 144 | 144 | 190 | 190 | 6.6 | 625 | Slight crystal |
| Recon w/ | (40° C.) | | | | | | | growth |
| NaCl | | | | | | | | Few particles |
| | | | | | | | | >5 um |
| LYO | 6 month | 136 | 136 | 178 | 178 | 6.6 | 603 | Slight crystal |
| Recon w/ | (25° C.) | | | | | | | growth |
| NaCl | | | | | | | | Few particles |
| | | | | | | | | >5 um |
| LYO | 6 month | 136 | 136 | 178 | 178 | 6.6 | 606 | Slight crystal |
| Recon w/ | (40° C.) | | | | | | | growth |
| NaCl | | | | | | | | Few particles |
| | | | | | | | | >5 um |
| GIL | Initial | 144 | 144 | 191 | 191 | 6.5 | 317 | Small particles, no |
| | | | | | | | | aggregation or |
| | | | | | | | | crystal growth |
| GIL | 2 week | 154 | 152 | 205 | 203 | 6.6 | 320 | Slight aggregation |
| | (25° C.) | | | | | | | |
| GIL | 2 week | 151 | 152 | 202 | 203 | 6.6 | 324 | Small particles, no |
| | (40° C.) | | | | | | | aggregation or |
| | | | | | | | | crystal growth |
| GIL | 4 Week | 151 | 150 | 200 | 196 | 6.6 | 314 | Crystal growth |
| | (25° C.) | | | | | | | ˜5 μm |
| GIL | 4 Week | 147 | 146 | 196 | 193 | 6.6 | 325 | Crystal growth |
| | (40° C.) | | | | | | | ˜5-10 μm |
| GIL | 8 Week | 141 | 140 | 188 | 186 | 6.6 | 322 | Crystal growth |
| | (25° C.) | | | | | | | ˜5 μm |
| GIL | 8 Week | 147 | 146 | 195 | 192 | 6.6 | 327 | Crystal growth |
| | (40° C.) | | | | | | | ˜5 μm |
| GIL | 12 Week | 148 | 146 | 196 | 194 | 6.6 | 634 | Slight crystal |
| Recon w/ | (25° C.) | | | | | | | growth |
| NaCl | | | | | | | | Few particles |
| | | | | | | | | >5 um |
| GIL | 12 Week | 143 | 142 | 191 | 189 | 6.6 | 622 | Slight crystal |
| Recon w/ | (40° C.) | | | | | | | growth |
| NaCl | | | | | | | | Few particles |
| | | | | | | | | >5 um |
| GIL | 6 month | 136 | 135 | 179 | 177 | 6.6 | 604 | Slight crystal |
| Recon w/ | (25° C.) | | | | | | | growth |
| NaCl | | | | | | | | Few particles |
| | | | | | | | | >5 um |
| GIL | 6 month | 139 | 139 | 185 | 183 | 6.6 | 608 | Slight crystal |
| Recon w/ | (40° C.) | | | | | | | growth |
| NaCl | | | | | | | | Few particles |
| | | | | | | | | >5 um |

*no son. = without sonication
**Recon w/NaCl = reconstitution with 0.9% sodium chloride

D. Particle Size and Optical Results

From Table 19 above, it is evident that the initial naproxen mean particle sizes of the LYO sample remained unchanged, with particle sizes of 142 nm & 144 nm, respectively, for the LYO and GIL samples. This was relatively comparable to the initial naproxen NCD particle size of 139 nm. After storage for 6 months at ambient and an elevated temperature of 40° C./75% RH, the mean particle sizes of both LYO and GIL samples remained unchanged at 136 nm and 139 nm, respectively.

Although the naproxen particle size as measured with the Horiba LA-910 Static Light Scattering Particle Analyzer yields consistent particle sizes over the course of the study, optical microscopy analysis showed physical instability in the form of possible naproxen crystal growth and aggregation at 2 months under both storage conditions. For this reason, 0.9% sodium chloride was used to reconstitute the samples to determine if this would aid in the physical stability.

Upon observation by optical microscopy, the samples appeared homogeneous and did not contain naproxen aggregates or large crystals. The naproxen particle sizes obtained for samples reconstituted with 0.9% sodium chloride were comparable to initial, 2 week, 1 month, and 2 month results. Reconstitution with 0.9% sodium chloride provides the desired physical stability.

E. Osmolality and pH Results

Osmolality values for the formulation are listed in Table 19. The osmolality values remained relatively constant in a range of 312 mOsm/kg to 334 mOsm/kg when stored for up to 2 months at ambient temperature and 40° C./75% RH. The osmolality increased at 3 and 6 months to between 602 mOsm/kg and 634 mOsm/kg for all temperatures and both LYO and GIL samples due to reconstituting with 0.9% sodium chloride. This is expected and is comparable to the theoretical osmolality of 540 mOsm/kg. The theoretical osmolality was calculated by including contributions from naproxen and PVP.

The initial pH for the NCD and LYO samples was 6.6. The pH at 2 months was 6.7, but decreased to 6.6 at subsequent time points. The initial pH of the GIL samples was 6.5, and 6.6 for all other timepoints (see Table 19). The slight changes are not an indication of instability because they are well within the variability limits of the pH meter.

F. Chemical Stability Results

Assay results of the LYO and GIL samples are illustrated in Table 20 below. The assay results were established by measuring the percent label claim (% LC) of samples of the by high performance liquid chromatography (HPLC). The HPLC assay procedure included preparing samples and standards at 1 mg/mL in 70:30 acetonitrile:H₂O. The mobile phase was 65% [0.05M KH₂PO₄ adjusted to pH 3 with H₃PO₄+1% of glacial acetic acid]: 35% acetonitrile. The chromatographic conditions were as follows: wavelength of detection: 270 nm; run time=20 min.; injection volume=10 μL; column temperature=25° C. The chromatographic system was a Waters 2690 Separations Module; Waters 2487 Dual Wavelength Detector; Waters Millennium 32 Chromatography Manager; and the column was a Brownlee RP8 Spheri-5 C8.

TABLE 20
Naproxen Percent Label Claim (% LC) &
Percent Degradation During 6 Month Stability

| | | Storage Temp. | % Label | Area % Degradation |
|---|---|---|---|---|
| Timepoint | Sample | (° C.) | Claim | Product |
| Initial | GIL | Ambient | 90.0 | 0.15 |
| Initial | LYO | Ambient | 90.5 | 0 |
| 2 Week | GIL | 25 | 95.1 | 0.14 |
| 2 Week | LYO | 25 | 95.5 | 0.00 |
| 4 Week | GIL | 25 | 95.1 | 0.16 |
| 4 Week | LYO | 25 | 95.1 | 0.00 |
| 8 Week | GIL | 25 | 94.6 | 0.13 |
| 8 Week | LYO | 25 | 95.0 | 0.00 |
| 12 Week | GIL | 25 | 96.6 | 0.30 |
| 12 Week | LYO | 25 | 97.0 | 0.00 |
| 6 Month | GIL | 25 | 92.4 | 0.17 |
| 6 Month | LYO | 25 | 92.7 | 0.00 |
| 2 Week | GIL | 40 | 95.4 | 0.14 |
| 2 Week | LYO | 40 | 96.2 | 0.00 |
| 4 Week | GIL | 40 | 119.4 | 0.16 |
| 4 Week | LYO | 40 | 96.0 | 0.00 |
| 8 Week | GIL | 40 | 93.8 | 0.30 |
| 8 Week | LYO | 40 | 95.4 | 0.00 |
| 12 Week | GIL | 40 | 96.8 | 0.21 |
| 12 Week | LYO | 40 | 94.1 | 0.01 |
| 6 Month | GIL | 40 | 92.5 | 0.23 |
| 6 Month | LYO | 40 | 93.1 | 0.00 |

Assay results at the initial timepoint are shown in Table 20. The LYO and GIL samples had initial label claims of 90.5% and 90%, respectively. Although the label claim was initially low, subsequent determinations indicate the label claim ranged from 92% to 97% over the course of the study. The 4 week 40° C./75% RH GIL data appears to be an outlier and is not consistent with the other data. Overall, the label claim is relatively stable at approximately 95%, with a slight downward trend at 6 months to approximately 93% for both pre- and post-gamma irradiation.

In this stability study, the main degradation product, Unknown 7, identified in Example 3 was monitored. This was the only degradation product which was evaluated because other degradation products are only present at insignificant levels. Quantitation was accomplished using the same HPLC procedure as above for % label claim and values are based on % peak area. Results are presented in Table 20 above.

The degradation product was initially absent in the LYO samples and present at 0.15% area in the GIL sample. Over the 6 month stability study, the average quantity of degradation product in the GIL samples was 0.18% area at 25° C. and 0.21% area for the 40° C./75% RH samples. Throughout the duration of the stability study, no degradation product was detected in the LYO samples stored at 25° C., and only 0.01% area for the 12 week sample stored at 40° C./75% RH. This provides further evidence this degradation product is generated upon gamma irradiation (see Example 3 for additional data).

The degradation product was identified by mass spectroscopy as a known naproxen API degradation product; 2-acetyl-6-methoxynapthalene.

Overall, the study indicates that the more concentrated naproxen formulation is physically and chemically stable over 6 months storage at 25° C. and 40° C./75% RH.

We claim:

1. A method for terminal sterilization of a solid form of a nanoparticulate active agent composition comprising:
(a) providing a solid form of at least one nanoparticulate active agent composition, wherein:
(1) the nanoparticulate active agent composition comprises at least one active agent and at least one surface stabilizer; and
(2) prior to formulation into a solid form the active agent has an effective average particle size of less than about 2 microns; and
(b) subjecting the solid form to gamma irradiation;
wherein said method produces a terminally sterilized solid form of at least one nanoparticulate active agent.

2. The method of claim 1, wherein said solid form is selected from the group consisting of tablets, capsules, dragees, trochees, sachets, lozenges, powders, pills, and granules.

3. The method of claim 2, wherein said powder is selected from the group consisting of lyophilized powders, spray dried powders, and spray granulates.

4. The method of claim 1, wherein said solid form is selected from the group consisting of a fast melt dosage form, controlled release dosage form, aerosol dosage form, lyophilized dosage form, delayed release dosage form, extended release dosage form, pulsatile release dosage form, mixed immediate release and controlled release dosage form, and a combination thereof.

5. The method of claim 1, wherein said solid form is formulated for administration via a method selected from the group consisting of oral, parenteral, pulmonary, nasal, rectal, vaginal, local, buccal, ocular, via the ear, and topical.

6. The method of claim 1, wherein the gamma irradiation is provided by applying a dose from about 5 to about 50 kGray.

7. The method of claim 1, wherein the gamma irradiation is provided by applying a dose from about 5 to about 25 kGray.

8. The method of claim 1, wherein the effective average particle size of the nanoparticulate active agent particles prior to formulation into a solid form is selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1 micron, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, and less than about 50 nm.

9. The method of claim 1, wherein upon redispersion of the sterilized solid form of at least one nanoparticulate active agent in a liquid media, the nanoparticulate active agent has an effective average particle size of less than about 2 microns.

10. The method of claim 9, wherein the redispersion media is selected from the group consisting of sterile water for injection, saline, dextrose, Lactated Ringer's solution, and Ringers solution.

11. The method of claim 9, wherein the redispersion media is a biorelevant media.

12. The method of any one of claims 9-11, wherein upon redispersion the nanoparticulate active agent has an effective average particle size selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1 micron, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, and less than about 50 nm.

13. The method of claim 1, wherein the concentration of at least one nanoparticulate active agent is selected from the group consisting of from about 99.5% to about 0.001%, from about 95% to about 0.1%, and from about 90% to about 0.5%, by weight, based on the total combined dry weight of the active agent particles and at least one surface stabilizer, not including other excipients.

14. The method of claim 1, wherein the concentration of the at least one surface stabilizer is selected from the group consisting of from about 0.001 to about 99.5%, from about 0.1 to about 95%, and from about 0.5 to about 90%, by weight, based on the total combined dry weight of the active agent particles and at least one surface stabilizer, not including other excipients.

15. The method of claim 1, wherein at least one active agent is in a form selected from the group consisting of crystalline particles, semi-crystalline particles, semi-amorphous particles, amorphous particles, and a mixture thereof.

16. The method of claim 1, wherein the active agent is selected from the group consisting of proteins, peptides, NSAIDS, COX-2 inhibitors, nutraceuticals, corticosteroids, elastase inhibitors, analgesics, anti-fungals, oncology therapies, anti-emetics, analgesics, cardiovascular agents, anti-inflammatory agents, anthelmintics, anti-arrhythmic agents, antibiotics, anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytics, sedatives, astringents, beta-adrenoceptor blocking agents, blood products and substitutes, cardiac inotropic agents, contrast media, corticosteroids, cough suppressants, diagnostic agents, diagnostic imaging agents, diuretics, dopaminergics, haemostatics, immunological agents, lipid regulating agents, muscle relaxants, parasympathomimetics, parathyroid calcitonin, biphosphonates, prostaglandins, radio-pharmaceuticals, sex hormones, anti-allergic agents, stimulants, anoretics, sympathomimetics, thyroid agents, vasodilators, and xanthines.

17. The method of claim 16, wherein the nutraceutical is selected from the group consisting of dietary supplements, vitamins, minerals, herbs, folic acid, fatty acids, fruit extracts, vegetable extracts, phosphatidylserine, lipoic acid, melatonin, glucosamine/chondroitin, Aloe Vera, Guggul, glutamine, amino acids, green tea, lycopene, whole foods, food additives, phytonutrients, antioxidants, flavonoid constituents of fruits, evening primrose oil, flax seeds, fish oils, marine animal oils, and probiotics.

18. The method of claim 1, wherein the active agent is selected from the group consisting of acyclovir, alprazolam, altretamine, amiloride, amiodarone, benztropine mesylate, bupropion, cabergoline, candesartan, cerivastatin, chlorpromazine, ciprofloxacin, cisapride, clarithromycin, clonidine, clopidogrel, cyclobenzaprine, cyproheptadine, delavirdine, desmopressin, diltiazem, dipyridamole, dolasetron, enalapril maleate, enalaprilat, famotidine, felodipine, furazolidone, glipizide, irbesartan, ketoconazole, lansoprazole, loratadine, loxapine, mebendazole, mercaptopurine, milrinone lactate, minocycline, mitoxantrone, nelfinavir mesylate, nimodipine, norfloxacin, olanzapine, omeprazole, penciclovir, pimozide, tacolimus, quazepam, raloxifene, rifabutin, rifampin, risperidone, rizatriptan, saquinavir, sertraline, sildenafil, acetyl-sulfisoxazole, temazepam, thiabendazole, thioguanine, trandolapril, triamterene, trimetrexate, troglitazone, trovafloxacin, verapamil, vinblastine sulfate, mycophenolate, atovaquone, atovaquone, proguanil, ceftazidime, cefuroxime, etoposide, terbinafine, thalidomide, fluconazole, amsacrine, dacarbazine, teniposide, and acetylsalicylate.

19. The method of claim 1 wherein the active agent is naproxen.

20. The method of claim 1, wherein the nanoparticulate active agent composition comprises at least two surface stabilizers.

21. The method of claim 1, wherein at least one surface stabilizer is selected from the group consisting of a nonionic surface stabilizer, an anionic surface stabilizer, a cationic surface stabilizer, and a zwitterionic surface stabilizer.

22. The method of claim 21, wherein at least one surface stabilizer is selected from the group consisting of cetyl pyridinium chloride, gelatin, casein, phosphatides, dextran, glycerol, gum acacia, cholesterol, tragacanth, stearic acid, stearic acid esters and salts, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hydroxypropyl methylcellulose, carboxymethylcellulose sodium, methylcellulose, hydroxyethylcellulose, hydroxypropylmethyl-cellulose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers, poloxamines, a charged phospholipid, dimyristoyl phophatidyl glycerol, dioctylsulfosuccinate, dialkylesters of sodium sulfosuccinic acid, sodium lauryl sulfate, alkyl aryl polyether sulfonates, mixtures of sucrose stearate and sucrose distearate, triblock copolymers of the structure: —(—PEO)—(—PBO—)—(—PEO—)—, p-isononylphenoxypoly-(glycidol), decanoyl-N-methylglucamide; n-decyl β-D-glucopyranoside, n-decyl β-D-maltopyranoside, n-dodecyl β-D-glucopyranoside, n-dodecyl β-D-maltoside, heptanoyl-N-methylglucamide, n-heptyl-β-D-glucopyranoside, n-heptyl β-D-thioglucoside, n-hexyl β-D-glucopyranoside, nonanoyl-N-methylglucamide, n-noyl β-D-glucopyranoside, octanoyl-N-methylglucamide, n-octyl-β-D-glucopyranoside, octyl β-D-thioglucopyranoside, lysozyme, a PEG derivatized phospholipid, PEG derivatized cholesterol, a PEG derivatized cholesterol derivative, PEG derivatized vitamin A, PEG derivatized vitamin E, and random copolymers of vinyl acetate and vinyl pyrrolidone.

23. The method of claim 21, wherein at least one cationic surface stabilizer is selected from the group consisting of a polymer, a biopolymer, a polysaccharide, a cellulosic, an alginate, a nonpolymeric compound, and a phospholipid.

24. The method of claim 21, wherein at least one surface stabilizer is selected from the group consisting of cationic lipids, benzalkonium chloride, sulfonium compounds, phosphonium compounds, quarternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅dimethyl hydroxyethyl ammonium chloride, C₁₂₋₁₅dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT 10™, tetrabutylammonium bromide, benzyl trimethylammonium bromide, choline esters, benzalkonium chloride, stearalkonium chloride compounds, cetyl pyridinium bromide, cetyl pyridinium chloride, halide salts of quatemized polyoxyethylalkylamines, MIRAPOL™, ALKAQUAT™, alkyl pyridinium salts; amines, amine salts, amine oxides, imide azolinium salts, protonated quaternary acrylamides, methylated quaternary polymers, cationic guar, polymethylmethacrylate trimethylammonium bromide, polyvinylpyrrolidone-2-dimethylaminoethyl methacrylate dimethyl sulfate, hexadecyltrimethyl ammonium bromide, poly (2-methacryloxyethyltrimethylammonium bromide) (S1001), poly(N-vinylpyrrolidone/2-dimethylaminoethyl methacrylate) di methylsulphate quarternary (S1002), and poly(2-methylacryloxyamidopropyltrimethylammonium chloride) (S1004).

25. A solid form of a nanoparticulate active agent composition made by the method of claim 1.

26. A terminally sterilized solid form of at least one nanoparticulate active agent comprising:
(1) at least one active agent, wherein prior to formulation into a solid form the active agent has an effective average particle size of less than about 2 microns; and
(2) at least one surface stabilizer; and
wherein said solid form has been subjected to gamma irradiation sufficient to terminally sterilize the solid form.

27. The solid form of claim 26, wherein said solid form is selected from the group consisting of tablets, capsules, dragees, trochees, sachets, lozenges, powders, pills, and granules.

28. The solid form of claim 27, wherein said powder is selected from the group consisting of lyophilized powders, spray dried powders, and spray granulates.

29. The solid form of claim 26, wherein said solid form is selected from the group consisting of a fast melt dosage form, controlled release dosage form, aerosol dosage form, lyophilized dosage form, delayed release dosage form, extended release dosage form, pulsatile release dosage form, mixed immediate release and controlled release dosage form, and a combination thereof.

30. The solid form of claim 26, wherein said solid form is formulated for administration via a method selected from the group consisting of oral, parenteral, pulmonary, nasal, rectal, vaginal, local, buccal, ocular, via the ear, and topical.

31. The solid form of claim 26, wherein the gamma irradiation is provided by applying a dose from about 5 to about 50 kGray.

32. The solid form of claim 26, wherein the gamma irradiation is provided by applying a dose from about 5 to about 25 kGray.

33. The solid form of claim 26, wherein the effective average particle size of the nanoparticulate active agent particles prior to formulation into a solid form is selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1 micron, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, and less than about 50 nm.

34. The solid form of claim 26, wherein upon redispersion of the sterilized solid form in a liquid media, the nanoparticulate active agent has an effective average particle size of less than about 2 microns.

35. The solid form of claim 34, wherein the redispersion media is selected from the group consisting of sterile water for injection, saline, dextrose, Lactated Ringer's solution, and Ringers solution.

36. The solid form of claim 34, wherein the redispersion media is a biorelevant media.

37. The solid form of any one of claims 34-36, wherein upon redispersion the nanoparticulate active agent has an effective average particle size selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1 micron, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, and less than about 50 nm.

38. The solid form of claim 26, wherein the concentration of at least one nanoparticulate active agent is selected from the group consisting of from about 99.5% to about 0.001%, from about 95% to about 0.1%, and from about 90% to about 0.5%, by weight, based on the total combined dry weight of the active agent particles and at least one surface stabilizer, not including other excipients.

39. The solid form of claim 26, wherein the concentration of the at least one surface stabilizer is selected from the group consisting of from about 0.001 to about 99.5%, from about 0.1 to about 95%, and from about 0.5 to about 90%, by weight, based on the total combined dry weight of the active agent particles and at least one surface stabilizer, not including other excipients.

40. The solid form of claim 26, wherein the active agent is in a form selected from the group consisting of crystalline particles, semi-crystalline particles, semi-amorphous particles, amorphous particles, and a mixture thereof.

41. The solid form of claim 26, wherein the active agent is selected from the group consisting of proteins, peptides, NSAIDS, COX-2 inhibitors, nutraceuticals, corticosteroids, elastase inhibitors, analgesics, anti-fungals, oncology therapies, anti-emetics, analgesics, cardiovascular agents, anti-inflammatory agents, anthelmintics, anti-arrhythmic agents, antibiotics, anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytics, sedatives, astringents, beta-adrenoceptor blocking agents, blood products and substitutes, cardiac inotropic agents, contrast media, corticosteroids, cough suppressants, diagnostic agents, diagnostic imaging agents, diuretics, dopaminergics, haemostatics, immunological agents, lipid regulating agents, muscle relaxants, parasympathomimetics, parathyroid calcitonin, biphosphonates, prostaglandins, radio-pharmaceuticals, sex hormones, anti-allergic agents, stimulants, anoretics, sympathomimetics, thyroid agents, vasodilators, and xanthines.

42. The solid form of claim 41, wherein the nutraceutical is selected from the group consisting of dietary supplements, vitamins, minerals, herbs, folic acid, fatty acids, fruit extracts, vegetable extracts, phosphatidylserine, lipoic acid, melatonin, glucosamine/chondroitin, Aloe Vera, Guggul, glutamine, amino acids, green tea, lycopene, whole foods, food additives, phytonutrients, antioxidants, flavonoid constituents of fruits, evening primrose oil, flax seeds, fish oils, marine animal oils, and probiotics.

43. The solid form of claim 26, wherein the active agent is selected from the group consisting of acyclovir, alprazolam, altretamine, amiloride, amiodarone, benztropine mesylate, bupropion, cabergoline, candesartan, cerivastatin, chlorpromazine, ciprofloxacin, cisapride, clarithromycin, clonidine, clopidogrel, cyclobenzaprine, cyproheptadine, delavirdine, desmopressin, diltiazem, dipyridamole, dolasetron, enalapril maleate, enalaprilat, famotidine, felodipine, furazolidone, glipizide, irbesartan, ketoconazole, lansoprazole, loratadine, loxapine, mebendazole, mercaptopurine, milrinone lactate, minocycline, mitoxantrone, nelfinavir mesylate, nimodipine, norfloxacin, olanzapine, omeprazole, penciclovir, pimozide, tacolimus, quazepam, raloxifene, rifabutin, rifampin, risperidone, rizatriptan, saquinavir, sertraline, sildenafil, acetyl-sulfisoxazole, temazepam, thiabendazole, thioguanine, trandolapril, triamterene, trimetrexate, troglitazone, trovafloxacin, verapamil, vinblastine sulfate, mycophenolate, atovaquone, atovaquone, proguanil, ceftazidime, cefuroxime, etoposide, terbinafine, thalidomide, fluconazole, amsacrine, dacarbazine, teniposide, and acetylsalicylate.

44. The solid form of claim 26 wherein the active agent is naproxen.

45. The solid form of claim 26 comprising at least two surface stabilizers.

46. The solid form of claim 26, wherein at least one surface stabilizer is selected from the group consisting of a nonionic surface stabilizer, an anionic surface stabilizer, a cationic surface stabilizer, and a zwitterionic surface stabilizer.

47. The solid form of claim 46, wherein at least one surface stabilizer is selected from the group consisting of cetyl pyridinium chloride, gelatin, casein, phosphatides, dextran, glycerol, gum acacia, cholesterol, tragacanth, stearic acid, stearic acid esters and salts, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hydroxypropyl methylcellulose, carboxymethylcellulose sodium, methylcellulose, hydroxyethylcellulose, hydroxypropylmethyl-cellulose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers, poloxamines, a charged phospholipid, dimyristoyl phophatidyl glycerol, dioctylsulfosuccinate, dialkylesters of sodium sulfosuccinic acid, sodium lauryl sulfate, alkyl aryl polyether sulfonates, mixtures of sucrose stearate and sucrose distearate, triblock copolymers of the structure: —(—PEO)—(—PBO—)—(—PEO—)—, p-isononylphenoxypoly-(glycidol), decanoyl-N-methylglucamide; n-decyl β-D-glucopyranoside, n-decyl β-D-maltopyranoside, n-dodecyl β-D-glucopyranoside, n-dodecyl β-D-maltoside, heptanoyl-N-methylglucamide, n-heptyl-β-D-glucopyranoside, n-heptyl β-D-thioglucoside, n-hexyl β-D-glucopyranoside, nonanoyl-N-methylglucamide, n-noyl β-D-glucopyranoside, octanoyl-N-methylglucamide, n-octyl-β-D-glucopyranoside, octyl β-D-thioglucopyranoside, lysozyme, a PEG derivatized phospholipid, PEG derivatized cholesterol, a PEG derivatized cholesterol derivative, PEG derivatized vitamin A, PEG derivatized vitamin E, and random copolymers of vinyl acetate and vinyl pyrrolidone.

48. The solid form of claim 46, wherein at least one cationic surface stabilizer is selected from the group consisting of a polymer, a biopolymer, a polysaccharide, a cellulosic, an alginate, a nonpolymeric compound, and a phospholipid.

49. The solid form of claim 46, wherein at least one surface stabilizer is selected from the group consisting of cationic lipids, benzalkonium chloride, sulfonium compounds, phosphonium compounds, quarternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅dimethyl hydroxyethyl ammonium chloride, C₁₂₋₁₅dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT 10™, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIRAPOL™, ALKAQUAT™, alkyl pyridinium salts; amines, amine salts, amine oxides, imide azolinium salts, protonated quaternary acrylamides, methylated quaternary polymers, cationic guar, polymethylmethacrylate trimethylammonium bromide, polyvinylpyrrolidone-2-dimethylaminoethyl methacrylate dimethyl sulfate, hexadecyltrimethyl ammonium bromide, poly (2-methacryloxyethyltrimethylammonium bromide) (S1001), poly(N-vinylpyrrolidone/2-dimethylaminoethyl methacrylate) di methylsulphate quarternary (S1002), and poly(2-methylacryloxyamidopropyltrimethylammonium chloride) (S1004).

50. A method of treating a mammal in need comprising administering the sterilized nanoparticulate active agent composition of claim 25.

51. A method of treating a mammal in need comprising administering the solid form of claim 26.

52. A method of treating a mammal in need comprising administering a liquid dosage form prepared by redispersing the solid form of claim 25 in a suitable liquid media.

53. A method of treating a mammal in need comprising administering a liquid dosage form prepared by redispersing the solid form of claim 26 in a suitable liquid media.
